(19) United States
(12) Patent Application Publication
(10) Pub. No.: US 2026/0240823 A1
(43) Pub. Date: Aug. 20, 2026

(54) METHODS OF TREATING, PREVENTING, AND/OR AMELIORATING CANCER WITH ACETYL COENZYME A (COA) CARBOXYLASE INHIBITOR(S)

(71) Applicant: Yale University, New Haven, CT (US)

(72) Inventors: Lajos Pusztai, New Haven, CT (US); Michal Marczyk, New Haven, CT (US)

(21) Appl. No.: 19/164,683

(22) PCT Filed: Mar. 13, 2024
(86) PCT No.: PCT/US2024/019660

§ 371 Date: Sep. 12, 2025

Related U.S. Application Data
(60) Provisional application No. 63/489,954, filed on Mar. 13, 2023.

(51) Int. Cl. A61K 31/438; A61K 31/137; A61K 45/06; A61K 47/68; A61P 35/00

(52) U.S. Cl. CPC A61K 31/438; A61K 31/137; A61K 45/06; A61K 47/68037; A61K 47/6851; A61K 47/6889; A61P 35/00

(57) ABSTRACT

The present disclosure relates, in one aspect, to a method of treating, preventing, and/or ameliorating cancer in a subject with administration of PF-05175157, or a salt or solvate thereof. In another aspect, the present disclosure relates to a method of treating preventing, and/or ameliorating cancer in a subject with administration of an acetyl-CoA carboxylase 1 and/or 2 inhibitor (ACCi) and at least one additional agent. In certain embodiments, the cancer is breast cancer.

### CROSS-REFERENCE TO RELATED APPLICATIONS

This application claims priority under 35 U.S.C. § 119(e) to U.S. Provisional Patent Application No. 63/489,954, filed Mar. 13, 2023, which is incorporated herein by reference in its entirety.

### BACKGROUND

Cancer cells undergo metabolic adaptation that enables rapid cell proliferation and survival in various tissue environments. Metabolic adaptation is driven by altered regulation of enzymatic functions by mutated oncogenes (e.g., PI3K and KRAS), changes in enzyme expression levels due to DNA copy number or epigenetic changes, and mutations that can alter substrate specificity. An important feature of metabolic rewiring is altered isozyme composition in cancer cells relative to normal cells. Isozymes are distinct proteins encoded by different genes that catalyze the same enzymatic reaction, but often with distinct intracellular localization, and different kinetics and substrate affinity. Most normal cells express diverse sets of isozymes, which contributes to the redundancy and robustness of critical biochemical processes, cancer cells often lose that diversity and express a single cancer-dominant isozyme. For example, of the four isozymes (PKL, PKR, PKM1, PKM2) of pyruvate kinase (PK) that catalyzes the final step of glycolysis, the L and R isoforms are expressed predominantly in the liver and red blood cells. the M1 isoform is common to most adult tissues, but in tumor tissues the M2 isoform (PKM2) is dominant.

It has not been systematically examined how many metabolic enzymes are affected by cancer specific loss of isozyme diversity (LID), which is defined as one isoform becoming the dominant isoform in cancer while corresponding normal tissues express a broad range of isoforms. LID could represent a potential therapeutic vulnerability of cancer cells if it affects a rate-limiting catalytic step in a cell survival critical biological process. It has been hypothesized that cancer cells may become dependent on a single isozyme to catalyze a critical metabolic step and that selective inhibition of such isozyme could lead to a metabolic bottleneck and cell death.

Acetyl-CoA carboxylase 1 and/or 2 (ACC1 and/or ACC2) catalyze the initial rate-limiting step of de novo fatty acid synthesis and is one of the most frequently affected enzymes by LID across a number of different types of cancer.

There is thus a need in the art for inhibitors of ACC1 and/or ACC2 and methods of use thereof for the treatment, prevention, and/or amelioration of cancer. The present disclosure addresses this need.

### BRIEF SUMMARY

In one aspect, the disclosure provides a method of treating, preventing, and/or ameliorating cancer in a subject in need thereof. In certain embodiments, the method comprises administering to the subject a dose of about 100 to about 600 mg of PF-05175157, or an equimolar amount of a pharmaceutically acceptable salt or solvate thereof.

In certain embodiments, the subject is further administered at least one additional agent. In certain embodiments, the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL1 84, kadcyla (ado-trastuzumab emtansine), lapatinib, larotrectinib, letrozole, margetuximab, megestrol, methotrexate, metformin, neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).

In another aspect, the disclosure provides a method of treating, preventing, and/or ameliorating cancer in a subject in need thereof. In certain embodiments, the method comprises administering to the subject a therapeutically effective amount of an acetyl-CoA carboxylase 1 and/or 2 inhibitor (ACCi) and at least one additional agent.

In certain embodiments, the ACCi is PF-05175157.

In certain embodiments, the subject is further administered at least one additional agent. In certain embodiments, the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib. JZL184, kadcyla (ado-trastuzumab emtansine), lapatinib, larotrectinib, letrozole, margetuximab, megestrol, methotrexate, metformin, neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).

### BRIEF DESCRIPTION OF THE FIGURES

The drawings illustrate generally, by way of example, but not by way of limitation, various embodiments of the present application.

FIGS. 1A-1F: characterization of isozyme mRNA expression in 14 cancer types. FIG. 1A: data sources and sample sizes for human enzymatic reactions (ENZYME database) and isozyme expression (The Cancer Genome Atlas ┌TCGA┐ data downloaded from RNAseqDB). FIG. 1B: number of paired tumor/normal samples, enzymatic reactions and isozymes included in the analysis of each cancer type. FIG. 1C: Venn diagram of shared and private enzymatic reactions between cancer types. FIG. 1D: top (red heat map): Median Pearson correlation coefficients of isozyme expression levels between patients within and across cancer types; bottom (blue heat map): Jaccard index of differentially expressed isozymes between tumor and corresponding normal tissues (defined as absolute log fold-change >2 and FDR <0.05). FIG. 1E: T-Distributed Stochastic Neighbor Embedding (t-SNE) plot based on expression levels of all isozymes (n=2,331) in all tumor and normal samples. FIG. 1F: proportion of differentially expressed isozymes between tumor and normal tissues. Each dot represents a patient, medians are indicated by the red vertical lines.

FIGS. 2A-2E: loss of isozyme diversity (LID) in cancer and identification of potential therapeutic isozyme targets. FIG. 2A: schematic representation of the strategy for finding an ideal therapeutic target isozyme showing LID. FIG. 2B: Venn diagram of private and shared enzymatic reactions showing LID across cancers. FIG. 2C: left bar graph: Total number of enzymatic reactions affected by LID in each cancer type, right table: Jaccard index of enzymes affected by LID across cancer types. FIG. 2D: linear regression line of average selection scores and their standard deviations (SD) across cancer types; each point represents an enzymatic reaction, the color scale (yellow to red) indicates the number of cancers in which it shows LID. Gray shading around the regression line indicates the 95% confidence intervals. FIG. 2E: over-representation analysis of LID enzymes in KEGG pathways. Color scale (yellow to red) reflects p-values from the enrichment test (*adjusted p<0.01, **adjusted p<0.001). Number of pathways affected in different cancer types are shown on sidebars.

FIGS. 3A-3C: prioritization of LID-based target isozymes by functional importance. FIG. 3A: number of LID-based target isozymes that satisfied the 3 criteria for the functional assessment by cancer type. Each row corresponds to different criteria. FIG. 3B: selection score distributions of isozymes that passed all 3 functional validation criteria and those that failed. FIG. 3C: association between number of cancer types in which isozymes showed LID (X-axis) and number of corresponding cancer cell line models in which the isozymes were functionally validated (Y-axis). Color scale shows percent functional validation rate, and the size of dots corresponds to the number of reactions.

FIGS. 4A-4E: identification and prioritization of potential therapeutic isozyme targets in breast cancer subtypes. FIG. 4A: Venn diagrams of shared and private LID-based isozyme targets in breast cancer subtypes; JI=Jaccard index comparing similarity between subtypes. FIG. 4B: over-representation analysis of LID enzymes in KEGG pathways. Color scale (yellow to red) reflects p-values from the enrichment test (*adjusted p<0.01, **adjusted p<0.001). Number of pathways affected in different subtypes are shown on sidebars. FIG. 4C: number of LID-based target isozymes that met 4 steps of the functional assessment by breast cancer subtype. FIGS. 4D-4E: normalized log 2 mRNA expression of ACC1, target isozyme and its non-target isoform ACC2 in 12 cancer types and corresponding normal tissues, respectively. Black lines show average values for each isozyme. BLCA: bladder urothelial carcinoma; BRCA: breast carcinoma; COAD: colon adenocarcinoma; ESCA: esophageal carcinoma: HNSC: head and neck squamous cell carcinoma; KICH: kidney chromophobe: LIHC: liver hepatocellular carcinoma; LUAD: lung adenocarcinoma; LUSC: lung squamous cell carcinoma; PRAD: prostate adenocarcinoma; STAD: stomach adenocarcinoma; and THCA: thyroid carcinoma.

FIGS. 5A-5E: inhibition of breast cancer cell viability and tumor growth by PF-05175157. FIG. 5A: dose response curve with 72 hours of exposure in 16 cell lines: red dotted lines indicate no effect of treatment; black frame highlights the normal epithelial cells. FIG. 5B: association between predicted maximum cell viability inhibition and log EC₅₀; black frame highlights normal epithelial cell line. FIG. 5C: proportion of cells in different cell cycle phases measured in 2 cell lines (rows) and 2 time points (columns). FIG. 5D: proportion of apoptotic cells measured in 2 cell lines (rows) and 2 time points (columns); significant differences between different doses (p<0.05) are marked with star. FIG. 5E: tumor growth curves in mouse MDA-MB468 xenograft and a patient-derived TNBC xenografts with and without (vehicle) drug treatment; lines indicate prediction of mixed-effect model. Error bars show 95% confidence intervals around mean values of the data.

FIGS. 6A-6D: in vitro transcriptomic changes induced by PF-05175157. FIG. 6A: volcano plots of differentially expressed genes at different time points in BT474 cells; red dots represent significantly affected genes (FDR <0.05 and log2FC >1). In the lower corners of each plot are the number of genes up- or down-regulated after treatment. FIG. 6B: validation of significantly altered genes at 24 hours in BT474 cells using data from MDAMB468 cells treated with PF-05175157 for 24 hours; red dots represent validated genes (i.e., P<0.05 in MDAMB468 cells and the same direction of change in both cell lines), blue dots represent not validated genes. FIG. 6C: gene set enrichment analysis using the Nanostring metabolic pathways; red indicates higher expression of gene set after treatment, blue indicates higher expression in control (* adjusted p<0.05. ** adjusted p<0.01). FIG. 6D: mRNA expression changes induced by PF-05175157 inhibition in key fatty acid metabolism enzymes mapped onto a schema of fatty acid metabolism.

FIGS. 7A-7C: in vitro metabolomic changes induced by PF-05175157. FIG. 7A: volcano plots show increased and decreased metabolites after PF-05175157 treatment of BT474 cells; red dots represent significantly affected metabolites (FDR <0.05 and log2FC >1); in the lower corners of each plot are the number of metabolites that increased or decreased after treatment. Metabolites are coded by KEGG compound ID. FIG. 7B: validation of metabolites significantly altered in BT474 cells using MDAMB468 cells; red dots represent validated metabolites (P<0.05 in MDAMB468 and the same direction of expression change in both cell lines), blue dots represent not validated metabolites. FIG. 7C: enrichment analysis of metabolites on KEGG metabolic pathways. Red indicates enrichment of a metabolic pathway after treatments (* adjusted p<0.05, ** adjusted p<0.01).

FIG. 8: inhibition of cell viability of the 4T1 mouse triple negative breast cancer cells by in vitro PF-05175157. The bar graphs depict cell viability normalized to vehicle (DMSO) exposure at different PF-05175157 concentrations and cell seeding densities. OMA (oligomycin) is positive control and the error bars show 95% confidence intervals around mean values of the data. Numbers in the table show % viability under different conditions.

FIG. 9A: inhibition of cell viability of the parental BT474 cells and two distinct clones of trastuzumab and pertuzumab resistant cells (BT-TPR1, BT-TPR2) by PF-05175157. The bar graphs depict cell viability normalized to vehicle (DMSO) exposure at different PF-05175157 concentrations. Resistant cells were generated from parental cells through serial culturing in the presence of increasing concentration of pertuzumab and trastuzumab. FIG. 9B: inhibition of cell viability of 4 different multiple myeloma cell lines in vitro by PF-05175157. The bar graphs depict cell viability normalized to vehicle (DMSO) exposure. Bortezumab is a positive control and the error bars show 95% confidence intervals around mean values of the data.

FIGS. 10A-10I: in vitro cell viability dose response curves for 5 different breast cancer cell lines (i.e., BT20, BT474, HCC1187, HCC1395, and MDAMB468) treated with a fixed dose of 10 μg/mL PF-05175157 and increasing doses of 9 partner drugs—ABT-510 (FIG. 10A), carboplatin (FIG. 10B), Danshensu (FIG. 10C), Doxorubicin (FIG. 10D), Gemcitabine (FIG. TOE), JZL184 (FIG. 10F). LOXO-101 (FIG. 10G), Metformin (FIG. 10H), and TVB-2640 (FIG. 10I). Gray lines indicate the single agent effect of the partner drug alone and red lines show cell viability for the PF-05175157 (10 μg/mL) plus partner drug combination (6 dose levels from 0 to 100 μM). The blue dotted line is the singe agent effect of PF-05175157 (10 μg/mL) in the given experiment. Error bars show 95% confidence intervals around mean values of triplicate wells. X-axis indicates μM concentrations of partner drugs.

FIG. 11A: human triple negative breast cancer patient derived xenograft (PDX) mouse tumor growth curves treated with single agent PF-05175157 administered orally twice daily and its solvent methvlcellulose administered the same way as control. FIG. 11B: tumor growth curves in mouse of MDA-MB468 human breast cancer cell line xenografts treated with different doses of PF-05175157 alone and in combination doxorubicin or vinorelbine. PF-05175157 was administered intraperitoneally once daily on days 1-5 followed by 2 days off FIG. 11C: tumor growth curves in mouse of HC1500 human estrogen receptor positive breast cancer cell line xenografts treated with PF-05175157 or fulvestrant (anti-estrogen therapy) or the combination of both. PF-05175157 was administered intraperitoneally (320 mg/kg) once daily on days 1-5, followed by 2 days off. Fulvestrant was administered (200 mg/kg) weekly subcutaneously. On all panels of FIGS. 11A-11C, error bars show 95% confidence intervals around mean values of tumor volume measurements in 6 mice per treatment group.

FIG. 12: tumor growth curves in mouse of fulvestrant resistant fluorescent labelled MCF-7 human estrogen receptor positive breast cancer cell line xenografts (orthotopic injection of 106 MCF7/FR-Luc transfected cells) treated with PF-05175157 or fulvestrant. Mouse also carried subcutaneous estradiol pallets. PF-05175157 was administered intraperitoneally 20 mg per kilogram once daily. Fulvestrant was administered 200 mg per kilogram twice weekly subcutaneously. Y-axis indicates florescent intensity at tumor implantation site obtained with IVIS Spectrum In Vivo Imaging System, error bars show 95% confidence intervals around mean values of tumor volume measurements in 5 mice per treatment group.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a.” “an.” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “ACC inhibitor” as used herein refers to a compound that inhibitors the activity of one or more acetyl-CoA carboxylase (ACC) enzymes (e.g., ACC1 and/or ACC2). In some embodiments, the ACC inhibitor is a compound that selectively inhibits the activity of ACC1. In other embodiments, the ACC inhibitor is a compound that selectively inhibits the activity of ACC2. In yet still other embodiments, the ACC inhibitor is a compound that inhibits the activity of both ACC1 and ACC2 (e.g., a dual, or non-selective, ACC inhibitor). Non-limiting examples of ACC inhibitors include PF-05175157, firsocostat, TOFA, ND-646, CMS-121, hACC2-IN-1. Acetyl-CoA Carboxylase-IN-1, CP-640186, MK-4074, olumacostat glasaretil, fluazifop-P-butyl, CP-640186, ACCT/2-IN-1, S-2E, ACC1/2-IN-2, CP-610431, A-908292, haloxyfop, and propaquizafop.

A disease or disorder is “alleviated” or “ameliorated” if the severity or frequency of at least one sign or symptom of the disease or disorder experienced by a patient is reduced.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “equimolar” refers to a quantitative comparison of the amount two chemical substances with regard to the number of moles, wherein the two chemical substances have the same number of moles, but not necessarily the same mass due to differences in the molar mass of the two chemical substances. For example, an approximately equimolar mixture of NaCl and KCl may comprise 58.4 μg of NaCl and 74.6 g of KCl, as the molar mass of NaCl and KCl are 58.443 g/mol and 74.551 g/mol, respectively.

The phrase “inhibit” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any one of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch: cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton. PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The terms “pharmaceutically effective amount” and “effective amount” refer to a non-toxic but sufficient amount of an agent to provide the desired biological result. That result can be reduction and/or alleviation of the signs, symptoms, or causes of a disease or disorder, or any other desired alteration of a biological system.

The terms “subject” or “patient” or “individual” for the purposes of the present disclosure includes humans and other animals, particularly mammals, and other organisms. Thus the methods are applicable to both human therapy and veterinary applications.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The terms “treat,” “treating.” and “treatment,” refer to one or more therapeutic or palliative measures described herein. The methods of “treatment” employ administration to a subject, in need of such treatment, a composition, for example, a subject afflicted with a disease or disorder, or a subject who has one or symptoms of such a disease or disorder, in order to cure, delay, reduce the severity of, or ameliorate one or more symptoms of the disorder or recurring disorder, or in order to prolong the survival of a subject beyond that expected in the absence of such treatment.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6.

### DESCRIPTION

PF-05175157 (1-isopropyl-1′-(2-methyl-1H-benzo[d]imidazole-6-carbonyl)-1,4-dihydrospiro[indazole-5,4′-piperidin]-7(6H)-one):

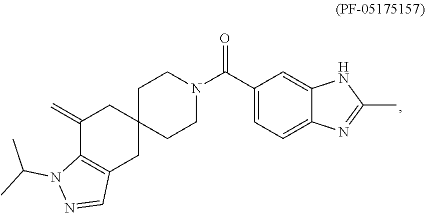 [SMILES: C=C1CC2(CCN(C(=O)c3ccc4nc(C)[nH]c4c3)CC2)Cc2cnn(C(C)C)c21]

is an investigational agent which is a potent and selective dual Acetyl-CoA carboxylase 1 and 2 (ACC1/ACC2) inhibitor that was originally developed as treatment for type 2 diabetes mellitus and acne vulgaris. Acetyl-CoA carboxylase (ACC) catalyzes the production of malonyl-CoA from acetyl-CoA. The malonyl-CoA generated by ACC serves two major functions in regulation of lipid metabolism. First, it is an essential and rate-limiting substrate for the de novo synthesis of fatty acids. Secondly, it inhibits the rate of beta-oxidation in the mitochondria through allosteric inhibition of carnitine-palmitoyl transferase-1 (CPT-1), which transports long-chain fatty acyl-CoAs into the mitochondria for oxidation. Inhibition or downregulation of ACC 1 has been shown to inhibit tumor growth in a broad range of in vitro and in vivo model systems. Therefore, in one aspect, the present disclosure relates to the discovery of the efficacy of PF-05175157 for the treatment of cancer in a subject. In certain embodiments, the cancer is breast cancer.

In another aspect, the present disclosure relates to the discovery of unexpected and/or synergistic results in the treatment of cancer, or biological assays indicative of efficacy in the treatment of cancer, upon administration of a combination of PF-05175157 and an additional agent.

In another aspect, the present disclosure relates to the discovery of unexpected and/or synergistic results in the treatment of cancer, or biological assays indicative of efficacy in the treatment of cancer, upon administration of a combination of an acetyl-CoA carboxylase 1 and/or 2 inhibitor (ACCi) and an additional agent.

### Methods

In one aspect, the present disclosure provides a method of treating, preventing, and/or ameliorating cancer in a subject in need thereof, the method comprising administering to the subject a therapeutically effective amount of PF-05175157, or a salt or solvate thereof.

In certain embodiments, the method of treating, preventing, and/or ameliorating cancer in a subject in need thereof comprises administering to the subject a dose of about 100 to about 600 mg of PF-05175157, or an equimolar amount of a pharmaceutically acceptable salt or solvate thereof.

In certain embodiments, the cancer is of the breast, bladder, blood, bone, bone marrow, brain, colon, esophagus, gastrointestine, gum, head, kidney, liver, lung, nasopharynx, neck, ovary, prostate, skin, stomach, testis, tongue, pancreas, and/or uterus.

In certain embodiments, the cancer is breast cancer.

In certain embodiments, the breast cancer is selected from the group consisting of HER2-negative metastatic breast cancer, ER-positive/HER2-negative breast cancer, and metastatic triple negative breast cancer (TNBC).

In certain embodiments, the PF-05175157 is administered orally.

In certain embodiments, about 200 mg of the PF-05175157 is administered to the subject twice daily. In certain embodiments, about 300 mg of the PF-05175157 is administered to the subject twice daily.

In certain embodiments, the daily administration is performed for 14 days with a subsequent period of 7 days without daily administration (e.g, 14 days treatment, 7 days without treatment, with repeating 21 day intervals).

In certain embodiments, the daily administration is performed for 7 days with a subsequent period of 7 days without daily administration.

In certain embodiments, subject is further administered at least one additional agent.

In certain embodiments, the at least one additional agent is a breast cancer therapeutic.

In certain embodiments, the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib. JZL184, kadcyla (ado-trastuzumab emtansine), lapatinib, larotrectinib, letrozole, margetuximab, megestrol, methotrexate, metformin, neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).

In certain embodiments, the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.

In certain embodiments, the at least one additional agent is elacestrant. In certain embodiments, the elacestrant is administered orally. In certain embodiments, about 400 mg of the elacestrant is administered to the subject daily.

In certain embodiments, the at least one additional agent is sacituzumab govitecan. In certain embodiments, the sacituzumab govitecan is administered intravenously. In certain embodiments, the subject is administered a dose of about 10 mg/kg of the sacituzumab govitecan daily.

In certain embodiments, the subject is a mammal. In certain embodiments, the mammal is a human.

In another aspect, the present disclosure provides a method of treating, preventing, and/or ameliorating cancer in a subject in need thereof, the method comprising administering to the subject a therapeutically effective amount of an acetyl-CoA carboxylase 1 and/or 2 inhibitor (ACCi) and at least one additional agent.

In certain embodiments, the ACCi is at least one selected from the group consisting of PF-05175157, firsocostat, TOFA, ND-646, CMS-121, hACC2-IN-1, Acetyl-CoA Carboxylase-IN-1, CP-640186, MK-4074, olumacostat glasaretil, fluazifop-P-butyl, CP-640186, ACC1/2-IN-1, S-2E, ACC1/2-IN-2, CP-610431, A-908292, haloxyfop, and propaquizafop.

In certain embodiments, the ACCi is PF-05175157.

In certain embodiments, the cancer is of the breast, bladder, blood, bone, bone marrow, brain, colon, esophagus, gastrointestine, gum, head, kidney, liver, lung, nasopharynx, neck, ovary, prostate, skin, stomach, testis, tongue, pancreas, and/or uterus.

In certain embodiments, the cancer is breast cancer.

In certain embodiments, the breast cancer is selected from the group consisting of HER2-negative metastatic breast cancer, ER-positive/HER2-negative breast cancer, and metastatic triple negative breast cancer (TNBC).

In certain embodiments, the PF-05175157 is administered orally.

In certain embodiments, about 200 mg of the PF-05175157 is administered to the subject twice daily. In certain embodiments, about 300 mg of the PF-05175157 is administered to the subject twice daily.

In certain embodiments, the daily administration is performed for 14 days with a subsequent period of 7 days without daily administration (e.g, 14 days treatment, 7 days without treatment, with repeating 21 day intervals).

In certain embodiments, the daily administration is performed for 7 days with a subsequent period of 7 days without daily administration.

In certain embodiments, subject is further administered at least one additional agent.

In certain embodiments, the at least one additional agent is a breast cancer therapeutic.

In certain embodiments, the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL184, kadcyla (ado-trastuzumab emtansine), lapatinib, larotrectinib, letrozole, margetuximab, megestrol, methotrexate, metformin. neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).

In certain embodiments, the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.

In certain embodiments, the at least one additional agent is elacestrant. In certain embodiments, the elacestrant is administered orally. In certain embodiments, about 400 mg of the elacestrant is administered to the subject daily.

In certain embodiments, the at least one additional agent is sacituzumab govitecan. In certain embodiments, the sacituzumab govitecan is administered intravenously. In certain embodiments, the subject is administered a dose of about 10 mg/kg of the sacituzumab govitecan daily.

In certain embodiments, the subject is a mammal. In certain embodiments, the mammal is a human.

### Pharmaceutical Compositions and Formulations

The disclosure provides pharmaceutical compositions comprising at least one ACC inhibitor (e.g., PF-05175157), which are useful to practice methods of the disclosure. Such a pharmaceutical composition may comprise at least one ACC inhibitor (e.g., PF-05175157), in a form suitable for administration to a subject, or the pharmaceutical composition may comprise at least one ACC inhibitor (e.g., PF-05175157), and one or more pharmaceutically acceptable carriers, one or more additional ingredients, or any combinations of these. The at least one ACC inhibitor (e.g., PF-05175157) may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier.

Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.010% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat, ameliorate, and/or prevent a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound: the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular embodiments, it is especially advantageous to formulate the compound in dosage unit form for ease of administration and uniformity of dosage. Dosage unit form as used herein refers to physically discrete units suited as unitary dosages for the patients to be treated: each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

Compounds of the disclosure for administration may be in the range of from about 1 μg to about 7,500 mg, about 20 μg to about 7,000 mg, about 40 μg to about 6,500 mg, about 80 μg to about 6.000 mg, about 100 μg to about 5,500 mg, about 200 p g to about 5,000 mg, about 400 μg to about 4.000 mg, about 800 μg to about 3.000 mg, about 1 mg to about 2,500 mg, about 2 mg to about 2,000 mg, about 5 mg to about 1.000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2.000 mg, or less than about 1.000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any one of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, and inhalational administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose: granulating and disintegrating agents such as cornstarch: binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White. 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling. forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example. 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6.340,475, 6,488.962, 6,451.808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXAMPLES

Various embodiments of the present application can be better understood by reference to the following Examples which are offered by way of illustration. The scope of the present application is not limited to the Examples given herein.

### Materials and Methods

### Pan-Cancer Human Gene Expression Data

RNA-seq expression data were obtained from the TCGA RNAseqDB database. Only cancer types with >10 paired tumor-normal samples were included in the analysis. Expression data was normalized using upper-quantile normalization and log 2 transformed (log 2(x+1)). For each cancer, only genes expressed in >25% of tumor-normal pairs or showed a ≥40% expression change in tumor versus normal were considered.

### Independent Breast Cancer Gene Expression Datasets

The E-GEOD-70951 data includes background-corrected, quantile normalized, and log 2 transformed microarray results generated on two different platforms (cohort 1 and cohort 2) from 195 paired breast cancer and normal breast tissues. A large batch effect between the two platforms and several isozymes of interest were not measured in cohort 1, therefore the validation analysis was restricted to cohort 2 of E-GEOD-70951 only, that included n=108 HR+/HER2−, n=10 HER2+, n=30 TNBC samples and matching normal tissues. The E-GEOD-76250 data includes normalized and log 2 transformed gene expression data from 33 paired TNBC and normal tissues.

### Enzyme Annotation

Information about enzymatic reactions and the isozymes were retrieved from the ENZYME nomenclature database, release October 2018 (enzyme dot expasy dot org), that contained 7,524 enzymatic reactions for which an enzyme commission (EC) number exists based on experimental data, and lists all isozymes that catalyze these reactions. Only the 1,341 enzymatic reactions that involve human proteins were considered.

### Definition of Loss of Isozyme Diversity (LID) and Ranking of Candidate Isozyme Targets

To detect enzymatic reactions with LID and to identify the most promising potential therapeutic targets, a three-step selection process was followed. First, enzymatic reactions catalyzed by only 2-5 isozymes were selected. Second, reactions for which only one isozyme was significantly upregulated, or unchanged, in cancer were identified, compared to corresponding normal tissue while all other isozymes catalyzing the same reaction showed lower expression in cancer. Third, isozymes were ranked based on how closely their expression characteristics approached an ideal LID expression profile using a target selection score that reflected five separate expression features, M1 through M5. M1 represents the expression of the target isozyme in cancer. M2 represents the expression of the complementary isozymes that catalyze the same reaction in cancer. M3 represents the difference between M1 and M2 (M3=M1−M2 in cancer) and quantifies the “cancer-dominance” of an isozyme. M4 quantifies the expression difference between the cancer dominant target isozyme and the complementary isozyme in corresponding normal samples (M4=target isozyme—complementary isozyme in normal tissue), it was assumed that the smaller M4 is, the less potential for toxic effects from a target isozyme specific inhibitor due to the higher expression of the complementary enzymes that would not be inhibited. M5 measures the difference between target isozyme dominance in cancer versus its dominance in normal tissues (M5=M3−M4), and it has been assumed that the larger the difference the greater the therapeutic index. These five measures were used to calculate a score that was the average of the sum of ranks of M1 through M5 for each candidate target isozyme (M2 and M4 were ranked in increasing order, while M1, M3 and M5 in decreasing order) presented in percentage scale and calculated separately in each cancer type.

### Cell Line Gene Expression Data and Gene Dependency Datasets

Cell line RNAseq data were obtained from the Cancer Cell Line Encyclopedia (CCLE, portals dot broadinstitute dot org/ccle, release of 8 Nov. 2017). The data was normalized using upper-quantile method and log 2 transformed (log 2(x+1)). In vitro functional importance of isozymes was estimated based on loss-of-function screens using RNAi (DEMETER2) and CRISPR (DepMap Public 19Q3). DEMETER2 contains screens for 17.309 genes in 712 cell lines. DepMap contains results for 17.634 genes in 563 cell lines. Both databases can be obtained through DepMap portal (depmap dot org/portal). Results for both screens are expressed as a gene dependency score (GD), which is defined as the effect of single-gene knock-down on cell viability after normalization and scaling to allow comparisons across difference cell lines and genes. A GD of 0 means no effect on cell viability, and negative values indicate decreased viability.

### In Vitro Cell Viability Assay

All cell lines analyzed in the study, BT20, BT474. BT483, BT549, HCC1143, HCC1187. HCC1395, HCC1500, HCC38. HCC70. HMEC. MDAMB231, MDAMB468, T47D and ZR75-1, were purchased from American Type Culture Collection (ATCC) and were cultured at 37° C., in 5% CO₂ in RPMI-1640 media (Gibco), except BT20 cells that were grown in EMEM media (Sigma), supplemented with 10% fetal bovine serum (Gibco). Three thousand cells were plated in each well of a 96 well plate and 24 hours later treated with various concentrations of PF-05175157 (Pfizer) or corresponding dimethyl sulfoxide (DMSO) diluent. Cell viability was determined using CellTitre-Glo Luminescent Cell Viability Assay (Promega, Madison. WI, USA) after 24, 48 and 72 hours of drug exposure. The assay reagent was added in equal amounts to the wells and cell lysis was performed at room temperature for 20 minutes. Luminescence was measured in a BioTek Synergy2 plate reader (BioTek, Winooski, VT, USA). The luminescence from the treated cells was compared to their respective untreated cells that served as a control for normalization. Dose response data were analyzed using dre R package by fitting symmetric log-logistic model. The main outputs of the model were: a lower limit of the response when the dose approaches infinity, the upper limit when the dose approaches 0, and EC₅₀, the dose required to reduce the viability half-way between the upper and lower limit.

### In Vitro Combinatorial Drug Screen Using FDA-Approved Anticancer Drugs

High throughput combinatorial drug screen was performed using Approved Oncology Drugs Set X (AOD X) available from Developmental Therapeutics Program at National Cancer Institute. The plate set contains 166 most current FDA-approved anticancer agents provided at 10 mM in 20 μL of 100% DMSO on Greiner 650201 96-well PP U-bottom plates. More information about the AOD X set can be found at https://dtp [dot] cancer [dot] gov/organization/dscb/obtaining/available_plates [dot] htm. Freshly prepared assay reagents and buffers were frozen at −80° C. in aliquots and used within a month of preparation. The assay was conducted in Corning 384-well, black, clear-bottom microtiter plates (Corning 3764). Compounds were first diluted in cell culture media to 11× concentration in intermediate plates from 10 mM library stocks using Beckman Coulter Echo 550 automated acoustic dispenser. Two (2) μL of 11× diluted compounds were then added to 20 μL of the seeded cells for the final concentrations of 0.05 μM, 0.1 μM, 1 μM and 10 μM using Beckman Biomek NXp. Finally, 0.2% DMSO, 0.2% DMSO with 10 μg/mL ACCi, and 60 μM Bortezomib were included as vehicle, negative and positive controls, respectively, using the Beckman Coulter Echo 550.

Mean and standard deviations of the control samples are used to calculate signal-to-background (S/B), coefficient of variation (CV), and Z′ values (Z′) for each screening plate to monitor assay performance and ensure rigor and screening quality. Mean values of the control samples were also used to normalize raw data for the test samples (on a per-plate basis) to enable data comparison across all test plates and experimental runs. Specifically, % toxicity for each test sample was calculated relative to the mean of the negative control samples (ACCi alone plus 0.2% DMSO vehicle) set as 0% toxicity and the mean of the positive control samples (60 uM Bortezomib) set as 100% toxicity. Unpaired two-tail student's t-test was used to evaluate the differences between ACCi single agent and combination treatments.

### Cell Cycle Analysis

Cell lines were plated in duplicate in 25 mm tissue culture dishes or in 6-well plates and treated 24 hours later with the indicated concentrations of PF-05175157. Docetaxel (10 nM, as a positive control) or with a DMSO control. At 24- and 72-hours post-treatment, cells were harvested and fixed overnight in 70% ethanol. Cells were then washed in PBS, stained with 50 μg/ml Propidium Iodide (Abcam) containing 550 U/mL RNase A, and assayed for DNA content on an LSR Fortessa Cytometer (BD Biosciences). Analysis was carried out using FlowJo software (BD Biosciences).

### Apoptosis Assay

Cell lines were plated in duplicate in 25 mm tissue culture dishes or in 6-well tissue culture plates and treated 24 hours later with the indicated concentrations of PF-05175157, Docetaxel (10 nM, as a positive control) or with a DMSO control. At 24- and 72-hours post-treatment, cells were harvested and stained with Annexin V-FITC according to the manufacturer's instructions (ANNEX300F, BioRad). Immediately prior to analysis by flow cytometry, Propidium Iodide (1 μg/mL) was added to the cells and assayed for apoptosis on an LSR Fortessa Cytometer (BD Biosciences). Analysis was carried out using FlowJo software (BD Biosciences). Annexin V single positive and Annexin V/PI double positive cells were considered apoptotic.

### Mouse Patient-Derived Xenograft (PDX) Models

The PDX experiments were performed by the In Vivo Services at The Jackson Laboratory Sacramento facility, according to an IACUC-approved protocol and in compliance with the Guide for the Care and Use of Laboratory Animals (National Research Council, 2011). The J000102184 patient derived xenograft model of triple negative breast cancer was tested in female NSG mice, NOD.Cg-Prkdc^{scid}Il2rg^{tm1Wjl}/SzJ (NSGTM: JAX 005557). Mice were housed in individually ventilated polysulfone cages with HEPA filtered air at a density of up to 5 mice per cage. The animal room was lighted entirely with artificial fluorescent lighting, with a controlled 12 h light/dark cycle (6 am to 6 pm light). The normal temperature and relative humidity ranges in the animal rooms were 22-26° C. and 30-70%, respectively. The animal rooms were set to have up to 15 air exchanges per hour. Filtered tap water, acidified to a pH of 2.5 to 3.0, and standard rodent chow was provided ad libitum. J000102184 tumor cells were injected into 7-week-old NSGTM mice at a volume of 40ul per mouse. Mice were enrolled when tumor volume reached between 100 to 200 mm³. Body weights, clinical observations and digital caliper measurements were recorded two times weekly post enrollment. Animals that reached a body condition score of ≤2, a body weight loss of ≥20%, ulcerated tumors, or a tumor volume >2000 mm³ were to be euthanized before study terminus.

On study day 0, dosing of mice began with PF-05175157. Group 1 was dosed with the vehicle comprised of 0.5% methycellulose in water. Group 2 was dosed with the 20 mg/kg of PF-05175157. Both groups were dosed twice daily by oral gavage for 4 weeks. On study day 43, tumor volumes were measured, and all animals were euthanized via CO₂ asphyxiation. At take-down, tumors from all mice were harvested for FFPE.

### Mouse Cell Line-Derived Xenograft (CDX) Models

Ten million MDAMB468 (ATCC@HTB132™) or HC1500 cells were implanted subcutaneously into the right flank of immune deficient Rag2/IL2RG double knockout mice (Envigo) in the presence of Matrigel (Corning). Twelve mice with palpable, similar sized tumors on post-implantation day 7 were randomized to two experimental groups: vehicle and PF-05175157 treatment. A 25 mg/mL stock of PF-05175157 was prepared by reconstitution in DMSO. The stock was diluted to 5 mg/mL in sterile PBS and administrated daily for five times a week by intraperitoneal injection in a 100 μL volume until the end of the experiment. Vehicle was prepared by diluting equal volume of DMSO in PBS. HC1500 xenografts were treated with PF-05175157 or fulvestrant (anti-estrogen therapy) or the combination of both. PF-05175157 was administered intraperitoneally 320 mg per kilogram once daily on days 1-5 followed by 2 days off Fulvestrant was administered 200 mg per kilogram weekly subcutaneously. In the MDAMB468 xenografts model intraperitoneal administration of PF-05175157 alone (80 mg/kg and 320 mg/kg once daily on days 1-5 followed by 2 days off) and in combination doxorubicin or vinorelbine were also assessed.

A fulvestrant resistant fluorescent labelled MCF-7 human estrogen receptor positive breast cancer cell line was orthotopically injected into SCID mice in 100 μL volume mixed with PBS and Matrigel (10⁶ MCF7/FR-Luc transfected cells) and subsequently treated with PF-05175157 or fulvestrant. Mouse also carried subcutaneous estradiol pallets. PF-05175157 was administered intraperitoneally 20 mg per kilogram once daily. Fulvestrant was administered 200 mg per kilogram twice weekly subcutaneously. Florescent intensity at tumor implantation site was assessed once a week using the IVIS Spectrum In Vivo Imaging System.

### Mouse Tumor Growth Curve Analysis

Both, CDX and PDX data were analyzed with mixed effect models using lme4 R package. There were two fixed effects in the model: day of study and type of drug used (Vehicle or PF-05175157), and a random effect allowing for random intercept for each animal to include individual variability in tumor volume. The interaction term between two fixed effects was added to model the effects of drug on the rate of tumor growth.

### RNA Sequencing Data Preparation and Analysis

RNA sequencing libraries were prepared from 1 μg of RNA using PolyA selection with oligo-dT beads, followed by random priming using the Illumina TruSeq Stranded Total RNA kit. Samples were sequenced with a target coverage of 50 million reads, paired-end, using the Illumina NovaSeq 6000 S4 platform. RNA sequencing was performed at the Yale Center for Genome Analysis. Three biological replicates were created in each scenario. Gene was called significant for FDR <0.05 and absolute value of log 2 fold change higher than 1. Gene was called validated for p-value <0.05 in independent cohort and the same direction of expression change.

### Metabolic Data Preparation and Analysis

Cells in each well were quenched by 150 μL of ice-cold quenching buffer. Quenching buffer recipe was as follows: 20% MEOH into 0.10% formic acid, 3 mM NaF, and 5.5 ug/mL d₈-phenylalanine (used as an internal standard). Then, the material was transferred to a LC/MS/MS V-bottom plate on dry ice, stored in −80° C. freezer till liquid is completely frozen and lyophilized overnight. The lyophilized powder was reconstituted in 50 μL/well d₄-taurine solution (another internal standard). Six biological replicates were created in each scenario. Each sample was run with two columns and two MS modes per column to get the best coverage of metabolites: reverse phase column with positive and negative MS mode: hypercarb column with positive and negative MS mode. The targeted group of metabolites was based on in-house IROA libraries (~600 metabolites in total) and the untargeted curation was based on the KEGG database (~2.700 metabolites). To call a metabolite significant, the same rules were applied as in the gene expression data analysis.

### Example 1: Isozyme Expression Patterns in Cancer and in Corresponding Normal Tissues

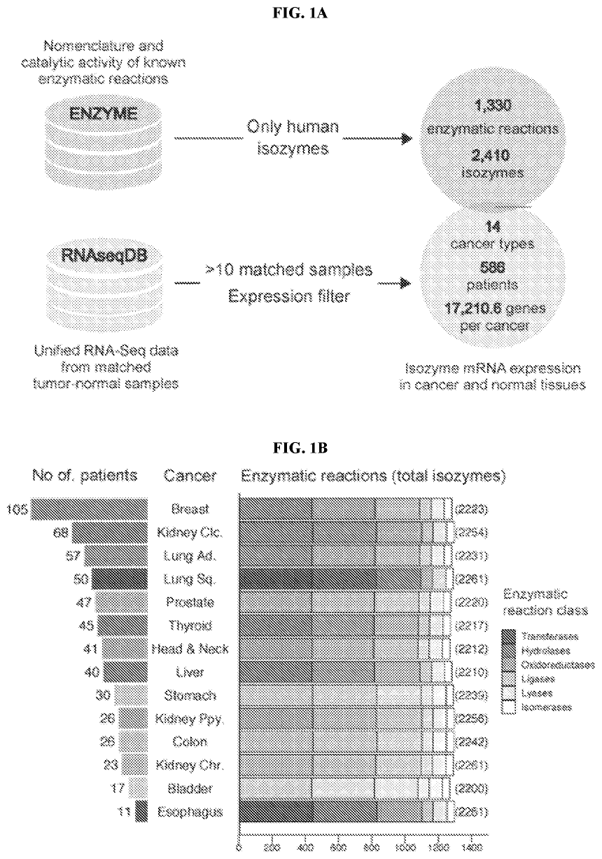

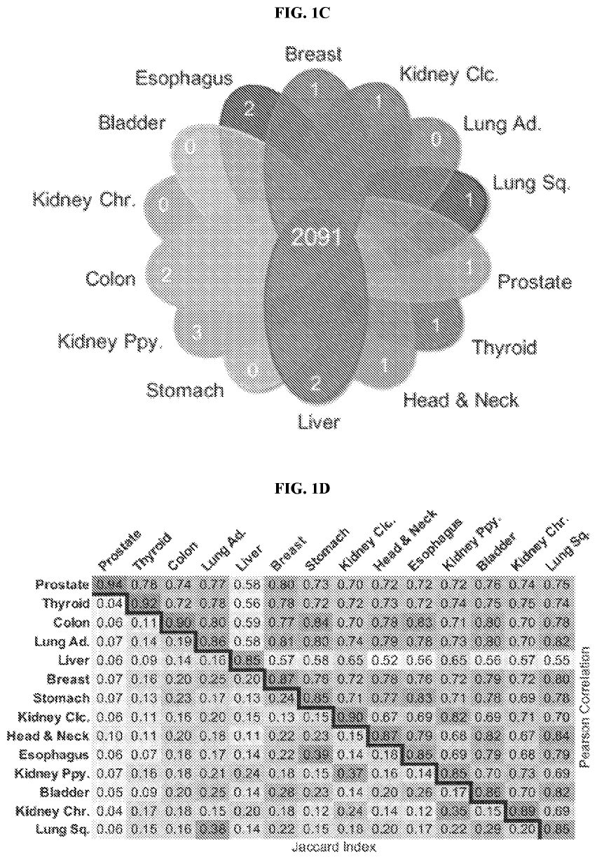

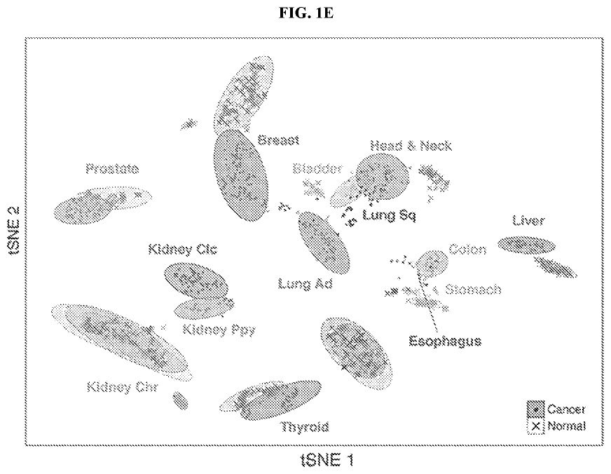

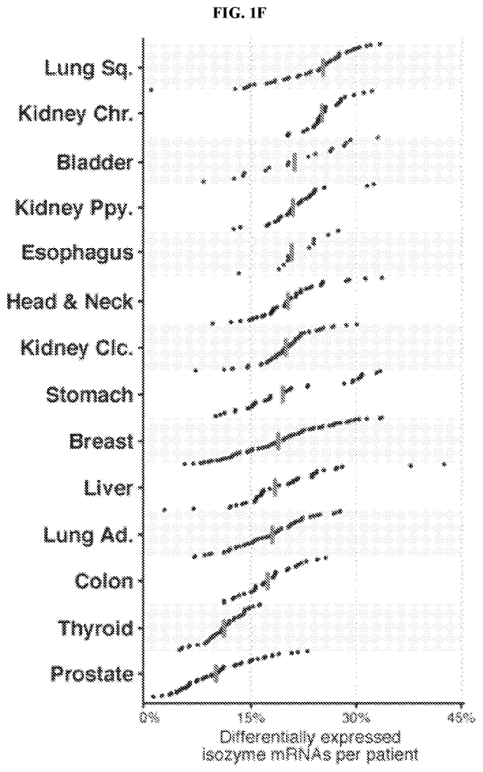

2,331 unique human isozymes catalyzing 1,319 different enzymatic reactions were identified in the ENZYME nomenclature database and obtained their mRNA expression data in 14 different cancer types and corresponding normal tissues from TCGA RNASeqDB database (FIG. TA). The number of available paired tumor-normal samples ranged from 11 in esophageal cancer to 105 in breast cancer (FIG. 1B). The overall number of expressed isozymes ranged from 2,200 to 2,261 across cancer types. 93% of enzymatic reactions and 90% of individual isozymes were detected in all cancer types, consistent with their fundamental roles in cell biology (FIGS. 1C-1D). Fifteen isozymes showed cancer type specific expression (FIG. TC).

Within each cancer type, the sample-to-sample median correlation coefficients for isozyme expression ranged from 0.85 to 0.94 indicating some between-cancer variation among histologically similar cancers. Differences in isozyme expression patterns were smaller between cancer and matching normal tissues than between different cancer types (FIG. 1E), indicating that most tissue-specific metabolic features persist in cancer cells.

Kidney and lung cancers showed the greatest difference from their corresponding normal tissues (FIG. 1E). Kidney chromophobe tumors also formed a cluster distinct from the other two types of kidney cancers and lung adenocarcinomas also separated from lung squamous cell carcinomas that clustered more closely with head and neck squamous cell cancers in the isozyme expression space (FIG. 1E). These observations are consistent with distinct cell types of origin for these cancer types. When differentially expressed isozymes were examined between cancer and corresponding normal tissue, it was found that most differentially expressed isozymes showed lower expression in cancer compared to normal tissues. Analyzing tumor-normal pairs at patient level, substantial variation in the proportion of differentially expressed isozymes from cancer to cancer in all cancer types was observed (FIG. 1F).

### Example 2: Loss of Isozyme Diversity in Cancer

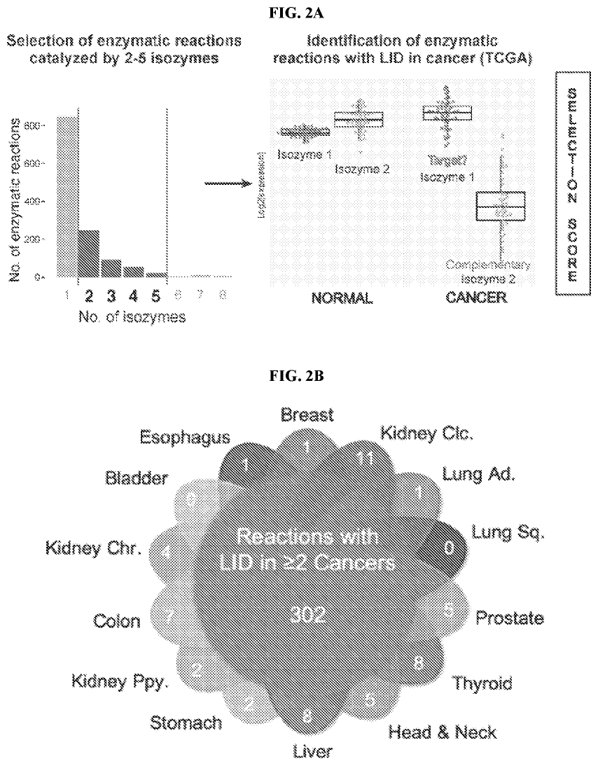

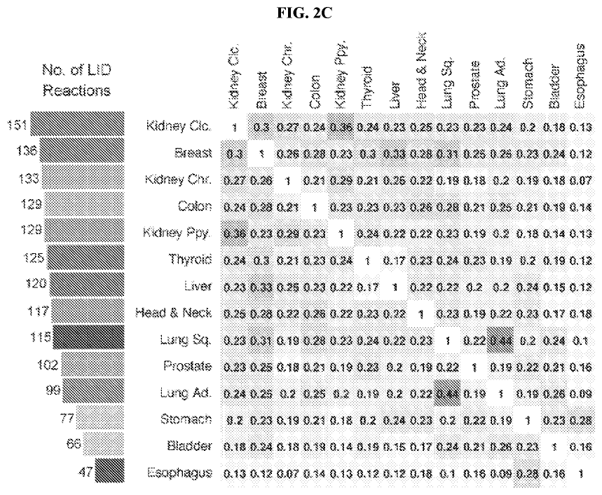

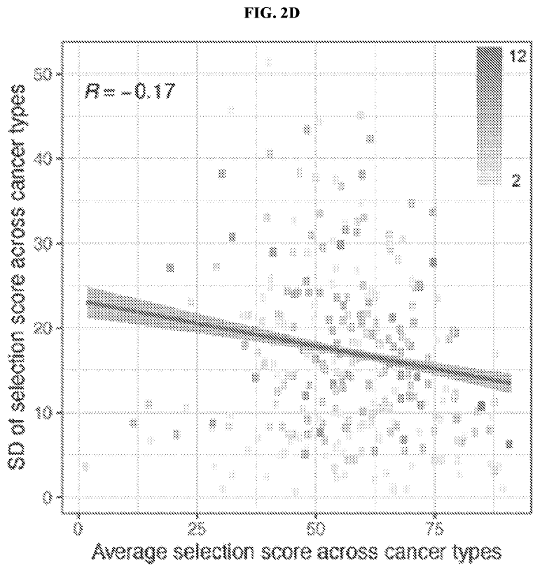

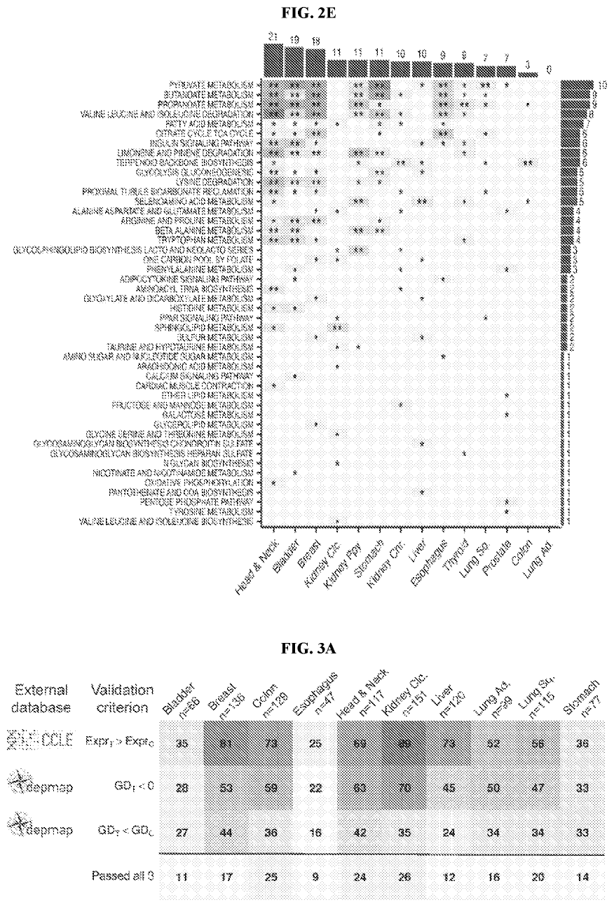

To identify isozymes affected by LID, the following selection criteria were applied (FIG. 2A): (i) the isozyme family includes 2-5 members that catalyze the same enzymatic reaction; (ii) one of the isozymes shows similar, or elevated, expression in cancer compared to normal (i.e., the cancer-dominant isoform) while the other isoforms (i.e., complementary isozymes) show lower expression in cancer compared to normal; and (iii) all isozyme members of the family show similar expression levels in normal tissue.

357 distinct enzymatic reactions were identified that met the selection criteria and showed LID in at least one cancer type. Fifty-five (15%) of these were observed only in a single cancer type (FIGS. 2B-2C). The number of LID-affected enzymatic reactions ranged from 47 in esophageal to 151 in kidney renal clear cell cancers (FIG. 2C). Across all cancer types, the most frequently LID-affected enzymatic reaction was EC 6.3.4.14/EC 6.4.1.2, catalyzed by acetyl-CoA carboxylase 1 (ACACA, also known as ACC1), which was the cancer-dominant isoform in 12 cancer types. The 357 LID-affected metabolic reactions represent a broad range of biochemical pathways (FIG. 2E). The top three KEGG pathways enriched for LID in 8 or more cancer types were all related to energy metabolism and biomolecule synthesis including pyruvate-, butanoate- and propanoate metabolism.

To rank the cancer-dominant LID-affected isozymes as potential therapeutic targets, a target selection score was next applied, which used five different features (M1 through M5) of their expression pattern that were combined into a single score which ranged between 0 to 100. Several isozymes showed consistently high selection scores across all cancer types (FIG. 2D), and three, including ACACA, L-dopachrome isomerase (MIF), and Beta-glucuronidase (GUSB), had uniformly high scores in more than 10 cancer types.

### Example 3: Expression of Candidate Isozyme Targets in Cancer Cell Lines and In Vitro Cell Viability Dependency from Genome-Wide Knockout Screens

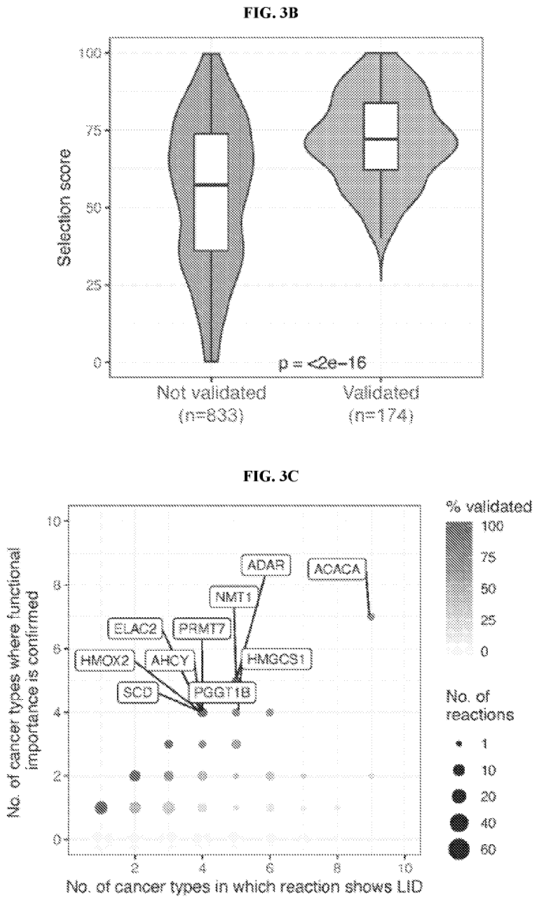

To assess the functional importance of the 357 cancer-dominant isozymes, their relevance for cell survival was ranked using gene dependency scores derived from in vitro whole-genome CRISPR and RNAi knockout datasets collated in DepMap and extracted isozyme expression levels for each cell line from the Cancer Cell Line Encyclopedia (CCLE). To prioritize the isozymes for functional relevance, the following 3 selection criteria were applied: (i) target isozyme expression is greater than the expression of the complementary isozymes in CCLE cancer cell lines (i.e., mimics expression pattern observed in corresponding tissues); (ii) target knockdown impairs tumor cell survival, as reflected by a negative gene dependency score; and (iii) target isozyme gene dependency score is lower than that of the complementary isozyme dependency score, implying a greater impact on cell survival. In this analysis, only cancer types for which 10 or more cell lines were available were included in the CCLE and DepMap databases (FIGS. 3A-3B).

Approximately 50% of the LID-affected isozymes showed cancer-dominant expression in the corresponding cell lines, but many were considered non-essential for cell viability based on dependency scores. The number of isozymes that satisfied all three functional prioritization criteria ranged from 9 in esophageal cancer to 26 in kidney clear cell carcinoma, resulting in an average functional validation rate of 17% (range 10% to 21%) across cancer types. Overall. 33 isozymes met the functional prioritization criteria in the cancer types in which they showed LID in the TCGA (FIG. 3C). The most broadly shared (i.e., showing LID in 9 cancer types) and functionally validated (in 7 cancer types in the CCLE/DepMap data) therapeutic target isozyme was ACACA.

### Example 4: Isozyme Targets in Breast Cancer Subtypes

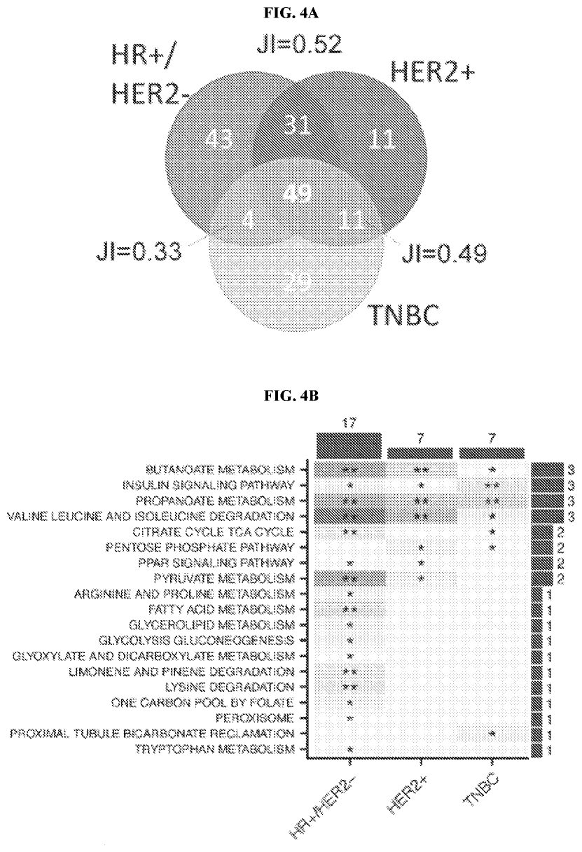

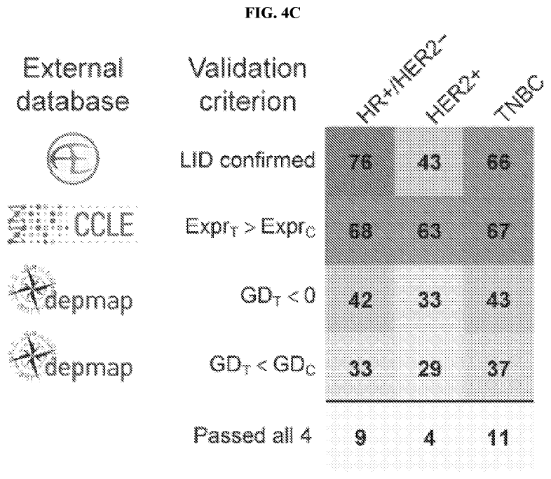

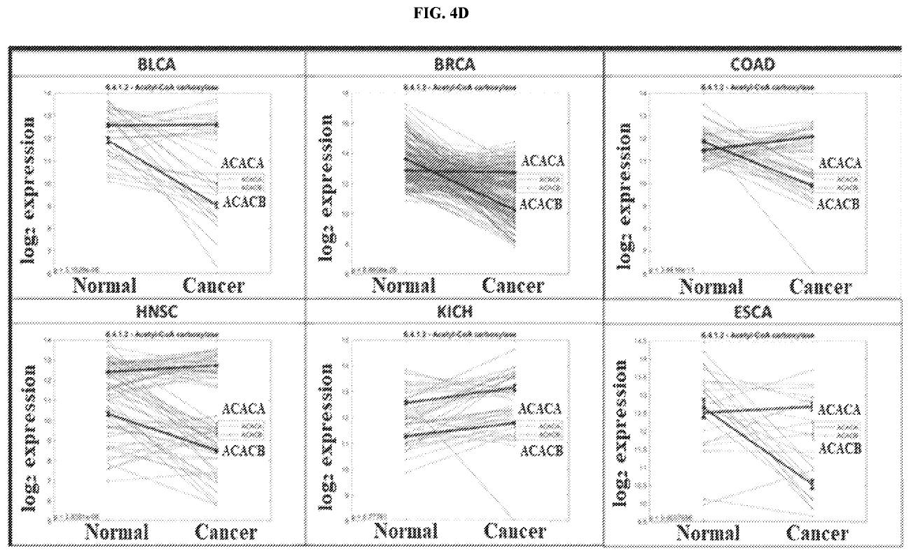

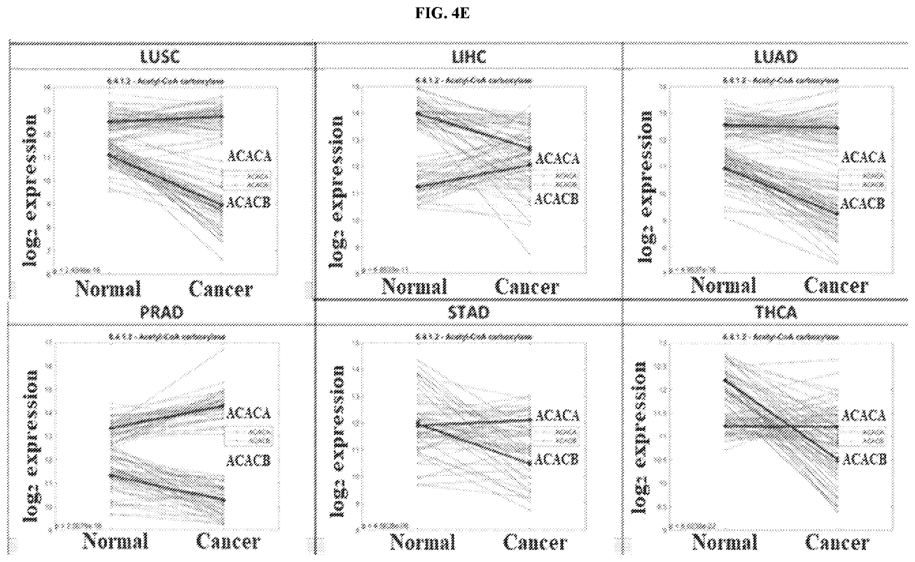

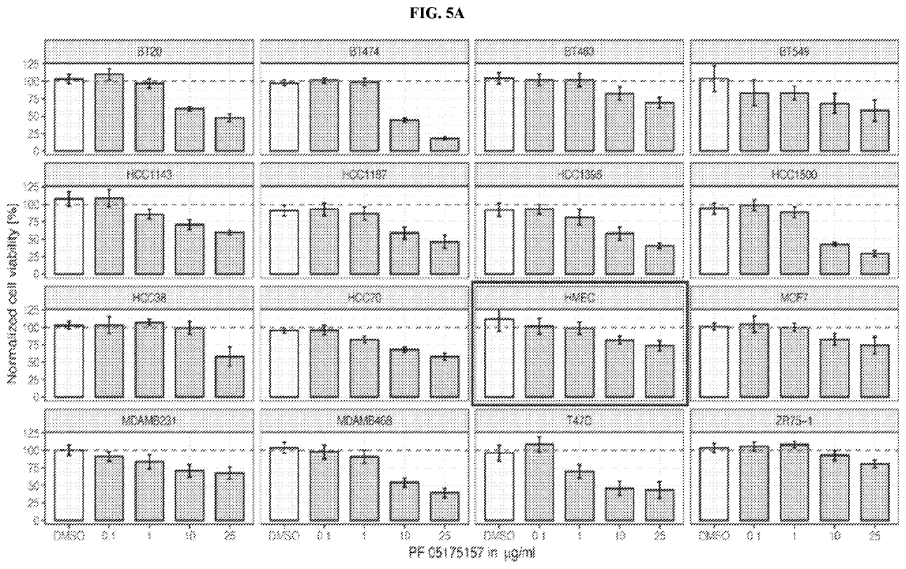

The largest single cancer type in the obtained data set was breast cancer. Since modem classification divides breast cancer into at least 3 clinically and molecularly distinct subtypes including hormone receptor positive/human epidermal growth factor receptor 2 negative (HR+/HER2−), HER2 positive (HER2+), and triple-negative (TNBC) cancers, this strategy was applied to discover potential isozyme targets for each of the distinct breast cancer subtypes. 127, 102 and 93 isozymes were identified showing LID in HR+/HER2−, HER2+, and TNBC subtypes, respectively. Forty-nine of the identified isozymes were cancer-dominant in all three subtypes, resulting in 178 unique potential metabolic targets (FIG. 4A). At the pathway level, carbohydrate metabolism pathways were the most enriched in therapeutic targets (FIG. 4B).

For breast cancer, in addition to the functional relevance criteria derived from the in vitro gene dependency analysis, and confirmed cancer-dominant expression in the CCLE, an additional gene expression-based validation step was introduced, which required confirmation of cancer-dominant expression of the target isozyme in two independent human breast cancer tissue mRNA expression datasets (E-GEOD-70951 and E-GEOD-76250). Nine isozymes met all criteria for high priority metabolic target in HR+/HER2− cancers. 4 for HER2+ cancers, and 11 for TNBC (FIG. 4C), corresponding to 17 unique isozymes. Across all cancer types, the most commonly LID affected isozyme was Acetyl-CoA-Carboxylase 1 (ACACA or ACC1) (FIGS. 4D-4E).

To explore potential mechanisms that could explain LID expression pattern of the 17 most promising isozyme targets in breast cancer, somatic mutation, copy number and methylation status of these genes were examined in the TCGA breast cancer subset that was used for the LID analysis (n=105 cases with matched normal and cancer tissue). Only 4 of the 17 genes had nonsvnonymous potentially deleterious mutations affecting 3 patients, and 8 of the 21 complementary isozymes carried mutations in 4 patients. Gene amplifications affected 6 target isozymes, most frequently observed in ADAR in 12 patients followed by ALDOA and CHKA in 7 patients each. Deletions affected 8 complementary isozymes but were rare, affecting only one or two patients each. When isozyme gene methylation status were compared on a continuous scale. 6 complementary isozymes (ACACB/ACC2, CHKB. PHGDH, ADARB1, POMT2, ELAC1) showed statistically significant hypermethylation compared to the corresponding target isozymes in all breast cancer subtypes. These findings suggest that methylation mediated gene silencing, rather than mutations or copy number alterations, are the most common cause of LID in breast cancer. ACC1 becomes cancer dominant due to silencing of ACC2 by gene methylation.

### Example 5: Effect of Small Molecule Inhibitor of ACACA/ACACB on Breast Cancer Viability In Vitro and In Vivo

ACACA was selected for functional validation because of its direct translational relevance. ACACA is a biotin-dependent enzyme that catalyzes the carboxylation of acetyl-CoA to produce malonyl-CoA, which is the rate-limiting step in de novo long-chain fatty acid synthesis. Pfizer has developed a drug, PF-05175157, that inhibits ACACA and ACACB with in vitro IC₅₀ concentrations of 27 nM and 33 nM, respectively. This drug has been tested in Phase I/II clinical trials in healthy volunteers and patients with type 2 diabetes (see NCT01819922, NCT01792635, and NCT01396161). These trials demonstrated significant inhibitory effect on lipid synthesis in patients and showed good pharmacokinetic properties with plasma Cₘₐₓ between 20-40 μg/mL.

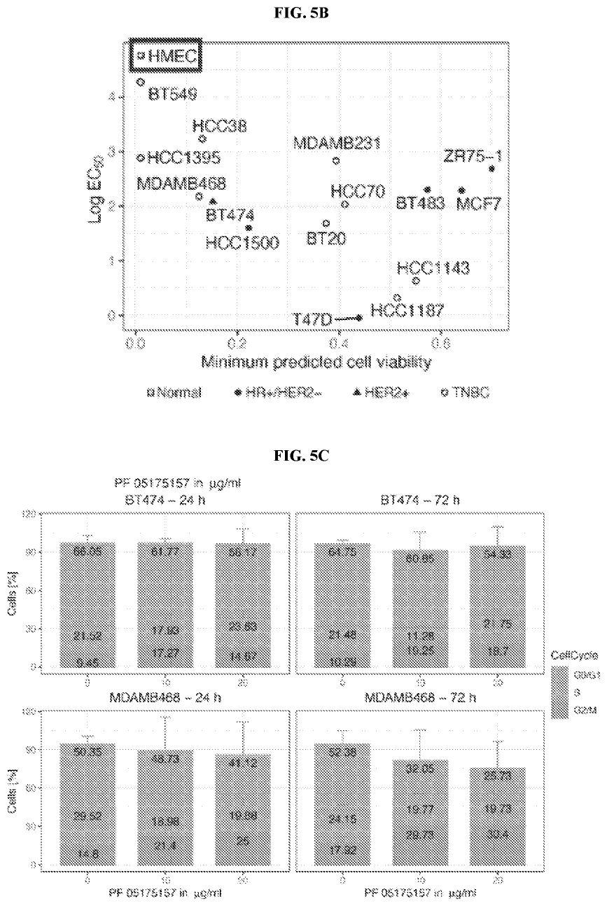

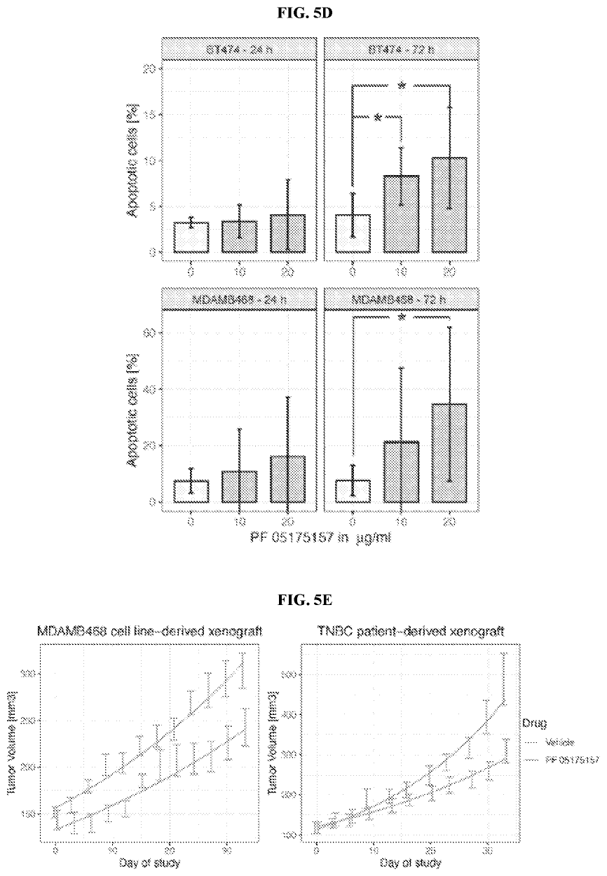

Breast cancer cells and normal breast epithelial (HMEC) cells were exposed to 0.1, 1, and 25 μg/mL of PF-05175157 or corresponding dilutions of DMSO for 24, 48 and 72 hours in vitro and observed statistically significant time- and dose-dependent inhibition of cell viability. A total of 15 breast cancer cell lines were tested and PF-05175157 at 10 μg/mL concentration significantly inhibited cell viability in all but 1 of the cell lines (FIG. 5A). Log-logistic model fitted to dose response curves gave different slopes, EC₅₀, and minimum viability for each cell line, but all measures of the inhibitory effect were highly significant. The estimated EC₅₀ ranged from 0.96 μg/mL in T47D to 66.9 μg/mL in BT549 cell line (FIG. 5B). The most resistant cells were HMEC with EC₅₀ of 117 μm/mL. Next, cell cycle progression and apoptosis were examined at 24 and 72 hours after exposure to PF-05175157 on the highly sensitive MDAMB468 and BT474 cells. There was a time dependent increase in cells at G2/M phase (FIG. 5C) followed by significant increase in apoptosis in both cell lines at 72 hours (FIG. 5D). The effect of ACACA inhibition may be rescued by lipid uptake from the surrounding tissues in therefore the efficacy of PF-05175157 was also assessed in MDAMB468 tumor xenografts (CDX; n=12) and a triple negative patient-derived xenograft (J000102184, PDX; n=10) model (FIG. 5E). In both models, PF-05175157 suppressed tumor growth compared to control (FIG. 5E).

### Example 6: Transcriptomic and Metabolic Profiling after ACACA/ACACB Inhibition in Breast Cancer Cell Lines

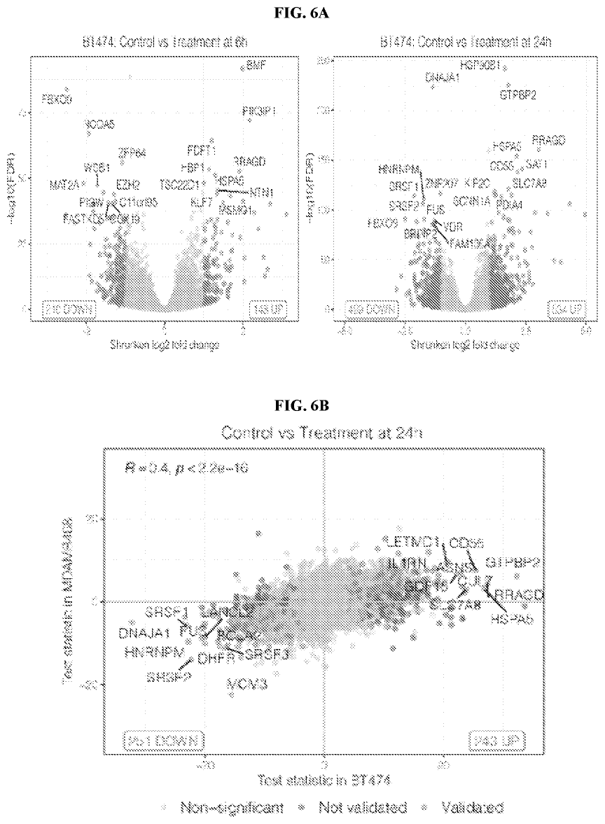

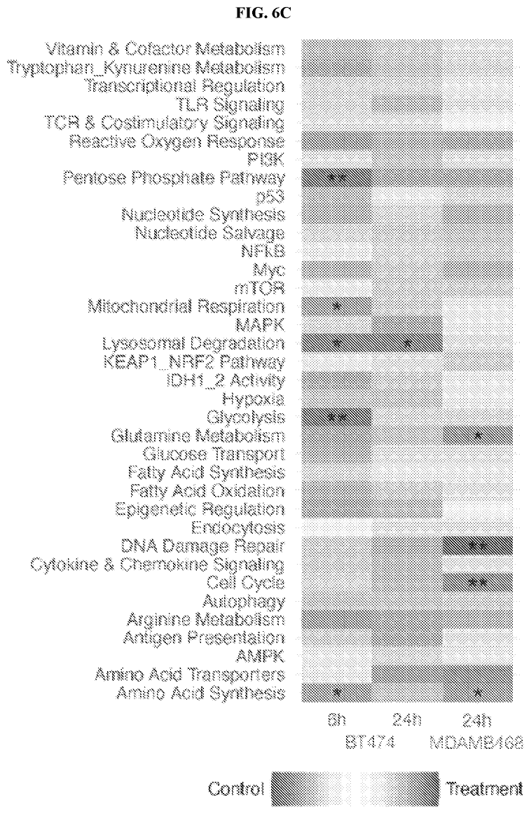

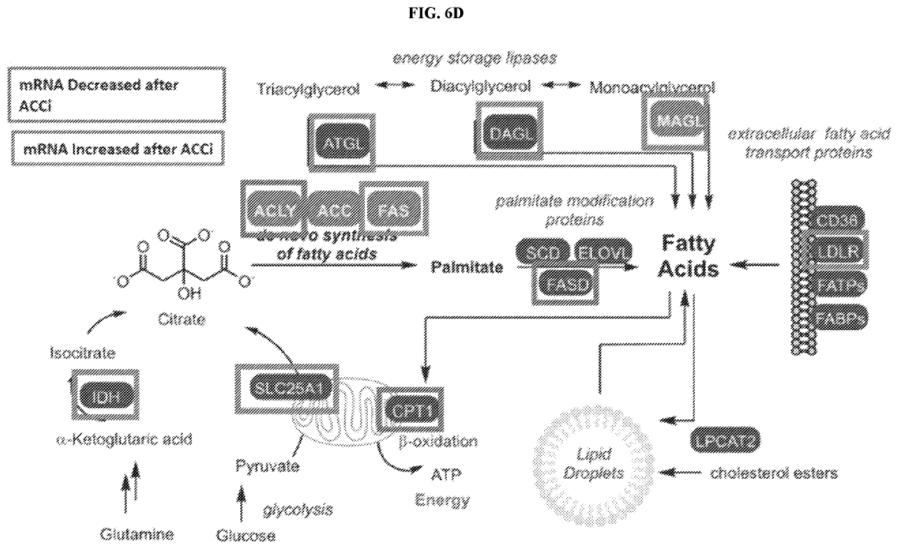

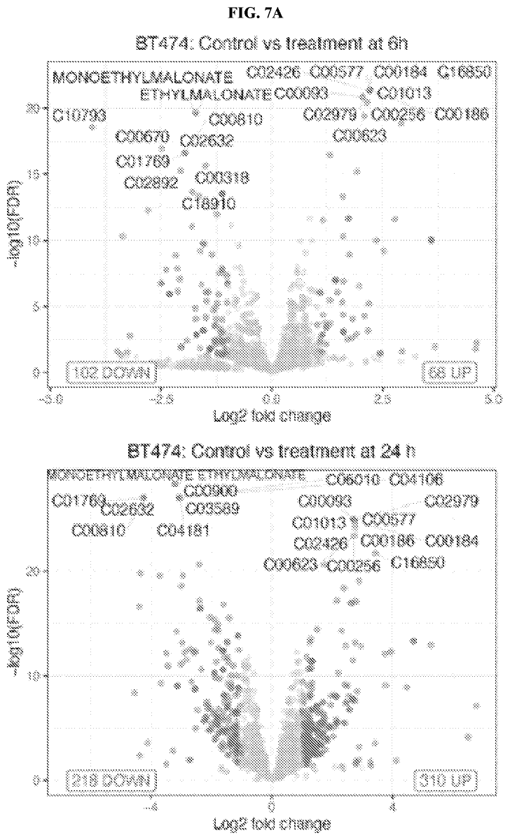

Transcriptomic and metabolomic changes were next examined after 6 and 24 hours of exposure to 10 μg/mL PF-05175157 in BT474 cells and after 24 hours exposure in MDAMB468 cells. In BT474 cells, at 6 hours, 148 genes were upregulated and 210 downregulated, and at 24 hours transcriptomic changes increased to 534 upregulated and 499 downregulated genes (FIG. 6A). The gene expression changes were highly correlated between the two time points (rₛ=0.66). Two hundred thirteen significantly upregulated and 251 downregulated genes at the 24-hour time point in the BT474 cells were also significantly upregulated in MDAMB468 cells, indicating a shared transcriptional response (FIG. 6B).

At pathway level, expression of many genes involved in metabolic processes was increased, suggesting a large scale metabolic compensatory response. Amino acid synthesis, glycolysis, lysosomal degradation, mitochondrial respiration, and pentose phosphate pathways showed a significant rapid upregulation at 6 hours (FIG. 6C). A significant decrease in DNA repair and cell cycle related genes was also observed by 24 hours in the MDAMB468 cells. A closer look at lipid metabolism showed significant upregulation of enzymes involved in fatty acid release from lipid stores (ATGL, DAGL, MAGL), fatty acid uptake from the extracellular space (LDLR) and citrate availability (IDH, SLC25A1) (FIG. 6D).

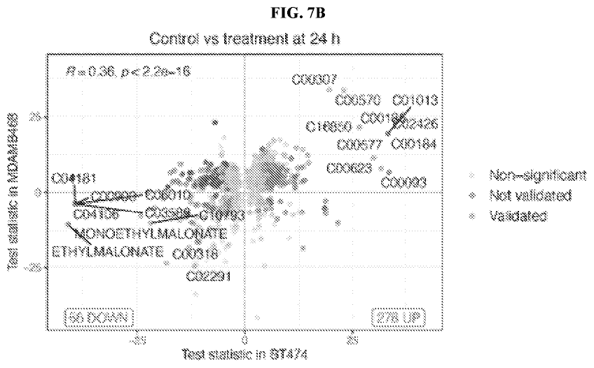

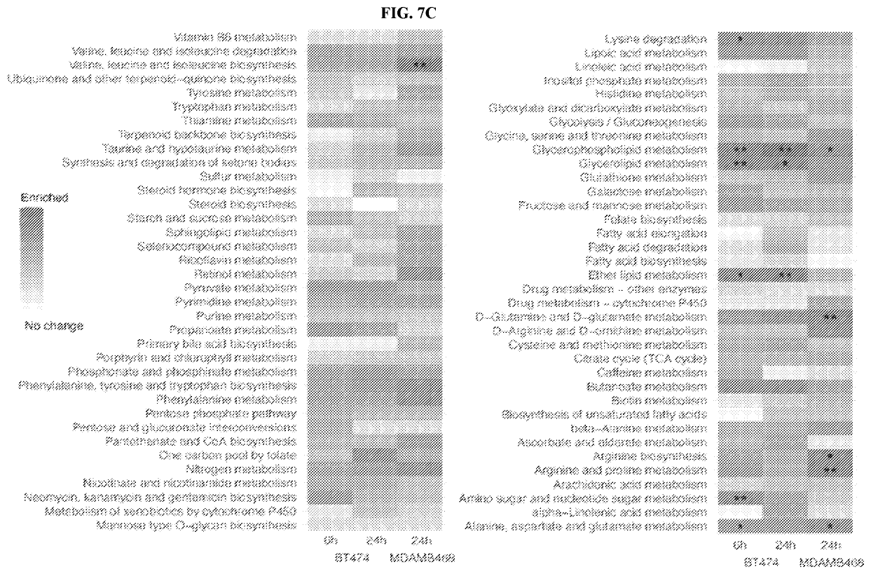

In contrast, an important regulator of fatty acid beta oxidation CPT1 decreased. These transcriptional changes suggest activation of processes that could provide alternate sources of fatty acids when de novo synthesis is inhibited (FIG. 6D). Metabolic profiling also showed extensive changes in metabolite levels across many metabolic pathways that tended to increase from 6 to 24 hours (FIG. 7A). Metabolite changes correlated over time indicating a consistent and sustained metabolic response (rₛ=0.71). Two hundred seventy-eight significant metabolites upregulated in BT474 cells, and 66 downregulated metabolites showed the same changes in MDAMB468 cells at the corresponding time points (FIG. 7B). At the metabolite abundance level, alterations in ether lipid, glycerolipid and glycerophospholipid metabolism were significant in both time points in BT474 cells, and similar changes were seen in MDAMB468 cells (FIG. 7C).

### Example 7: ACC Inhibition Studies

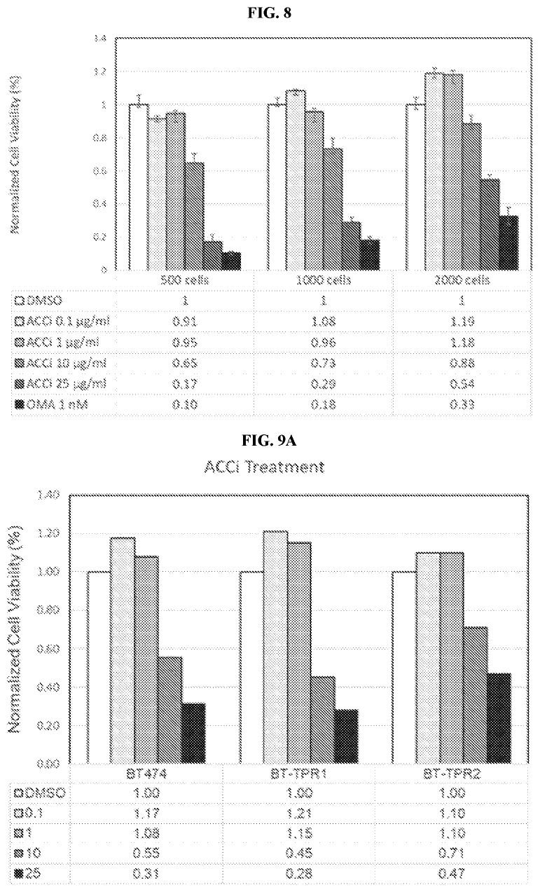

The efficacy of PF-05175157 was also assessed using a 4T1 mouse triple negative breast cancer cell line in vitro. Significant dose dependent inhibition was observed after 72 hours of exposure (FIG. 8). These results suggest that PF-05175157 also inhibits mouse ACC.

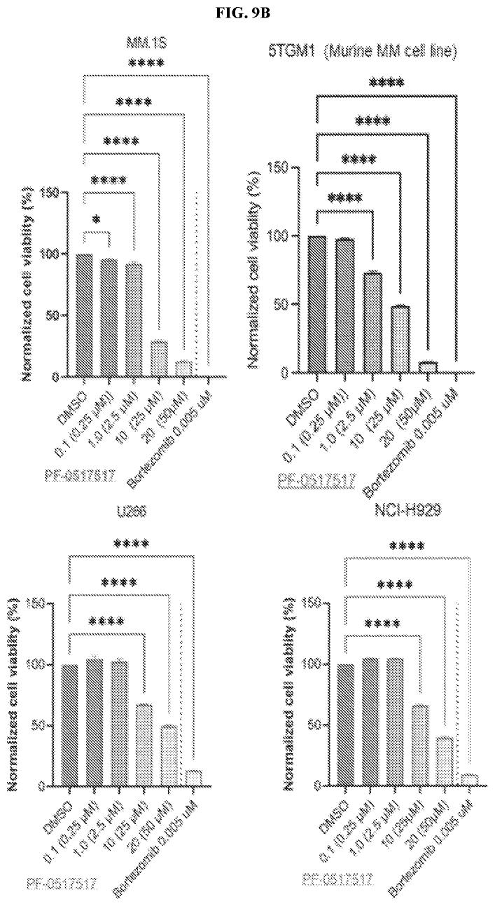

The efficacy of PF-05175157 was further evaluated using two distinct clones of trastuzumab and pertuzumab resistant cells derived from the HER2 amplified human BT474 human breast cancer cells. Significant in vitro growth inhibition was also demonstrated in these assays after 72-hour exposure in both the parental and HER2− targeted therapy resistant cells (FIG. 9A). These results indicate that ACC inhibition remains an effective way to suppress cells that become HER2− signaling independent. The efficacy of PF-05175157 was also evaluated in 4 myeloma cell lines in vitro, and in all 4 models, a dose-dependent inhibition was observed after 72 hour exposure (FIG. 9B).

A high throughput in vitro cell line screen including over 500 human cancer cell lines was performed to assess singe agent PF-05175157 activity beyond breast cancer cells. Cell lines with calculated IC₅₀ less than 10 μM (~4 μg/mL) are provided herein. The results are consistent with reports in the literature that ACC inhibition impairs cell viability in a broad range of cancers.

TABLE 1
Human cancer cell lines with PF-05175157 IC₅₀ < 10 μM
(~4 μg/mL)

| Cell | | IC₅₀ | Cell | | IC₅₀ |
|---|---|---|---|---|---|
| Line | TORClass | (μM) | Line | TORClass | (μM) |
| KMS-34 | Myeloma | 0.0032 | RPMI- | Myeloma | 4.257202 |
| | | | 8226 | | |
| MOLP-8 | Myeloma | 0.0032 | RMG-1 | Ovarian | 4.271178 |
| KMS-20 | Myeloma | 0.0032 | CAMA- | Breast | 4.76837 |
| | | | 1 | | |
| KMS- | Myeloma | 0.0032 | HCC1419 | Breast | 4.823103 |
| 12BM | | | | | |
| NCI- | Myeloma | 0.0032 | SNU- | Liver | 6.278542 |
| H929 | | | 182 | | |
| S34 | Sarcoma | 0.0032 | T-47D | Breast | 6.820009 |
| A 498 | Kidney | 0.0032 | H2342 | Lung | 7.289272 |
| KASUMI- | AML | 0.0032 | H1563 | Lung | 7.830864 |
| 1 | | | | | |
| KMS-27 | Myeloma | 0.456799 | U266B1 | Myeloma | 8.491161 |
| JIMT-1 | Breast | 0.475109 | H661 | Lung | 9.273671 |
| EN | Endometrial | 1.539322 | MM.1R | Myeloma | 9.431518 |
| LP-1 | Myeloma | 2.02791 | OVK- | Ovarian | 9.775558 |
| | | | 18 | | |
| HI1944 | Lung | 2.574997 | BIRCH | Sarcoma | 9.911089 |
| PLC/ | Liver | 3.939012 | JHH-4 | Liver | 9.999818 |
| PRF5 | | | | | |

### Example 8: PF-05175157 Anticancer Activity in Combination with Other Anticancer Therapies In Vitro and In Vivo

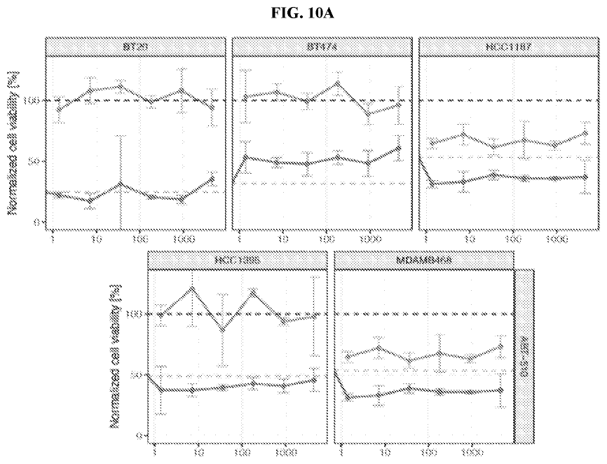

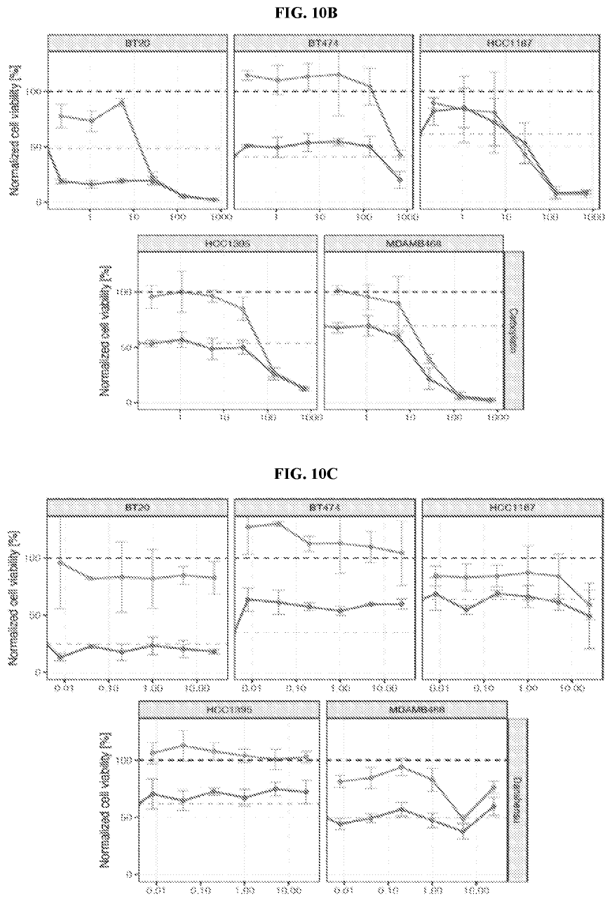

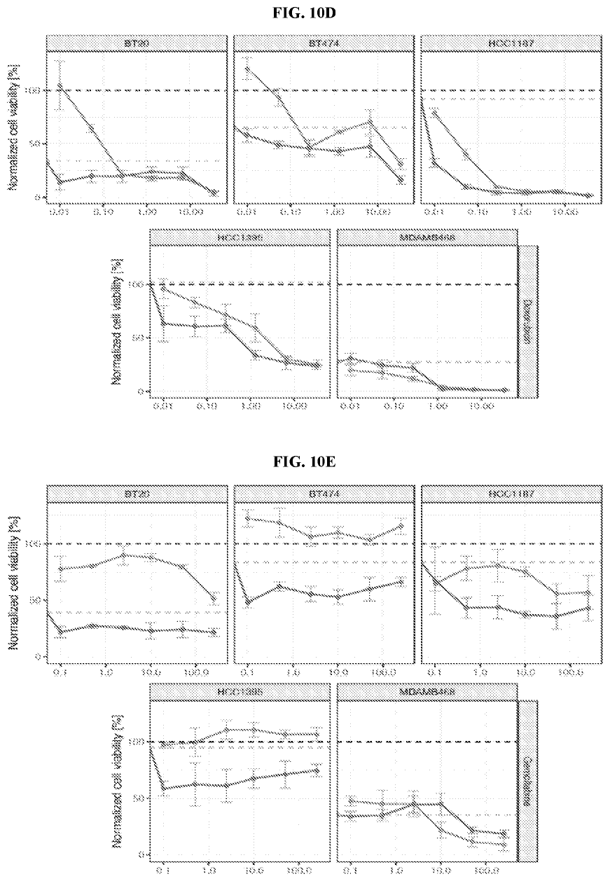

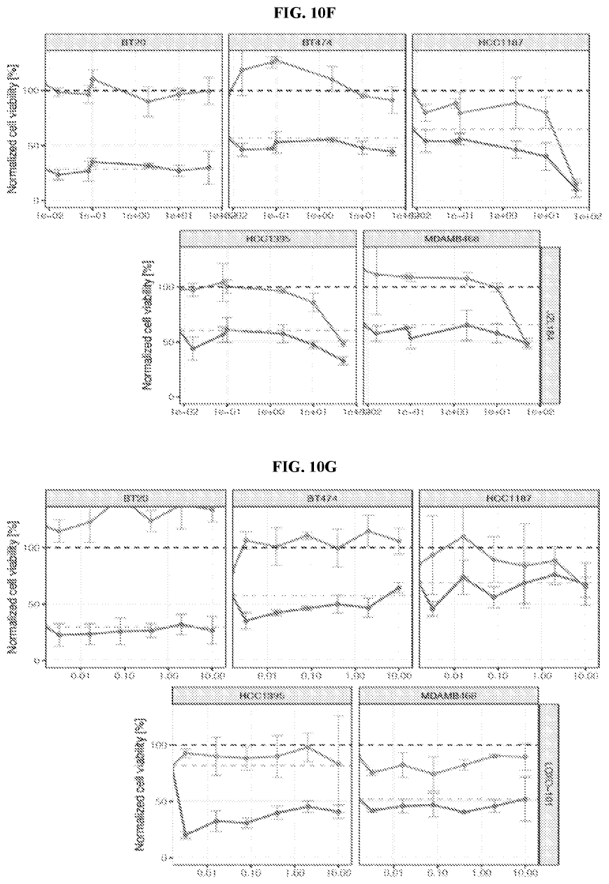

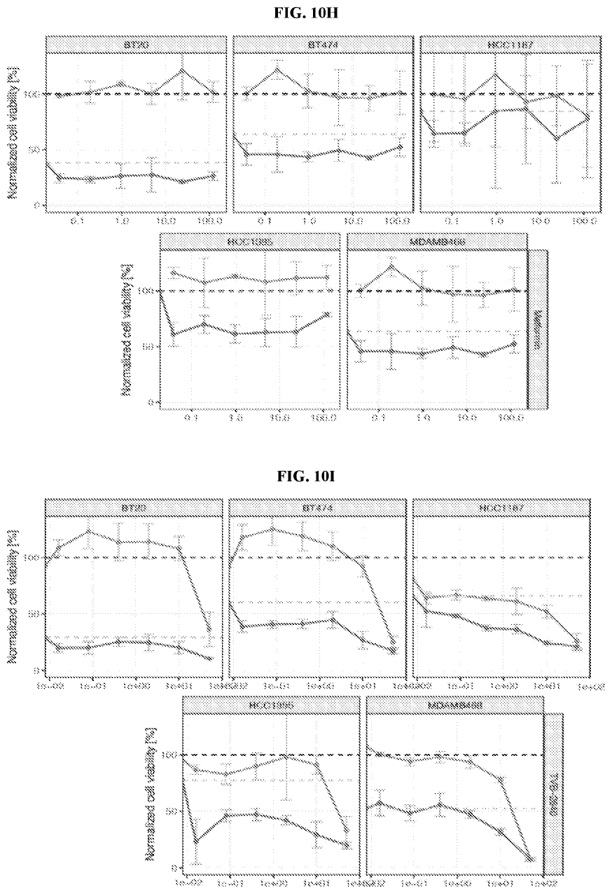

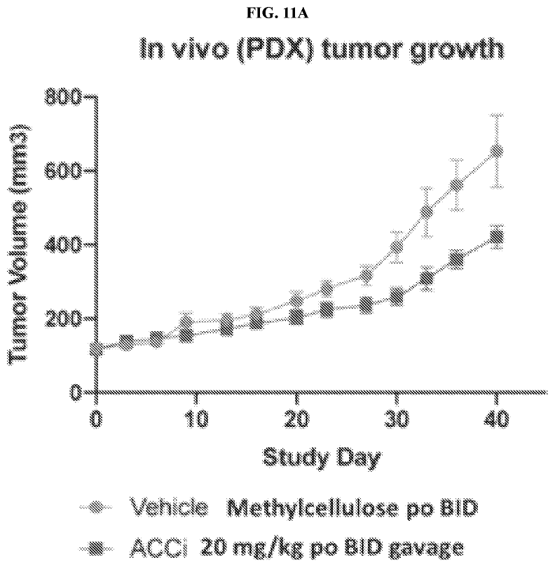

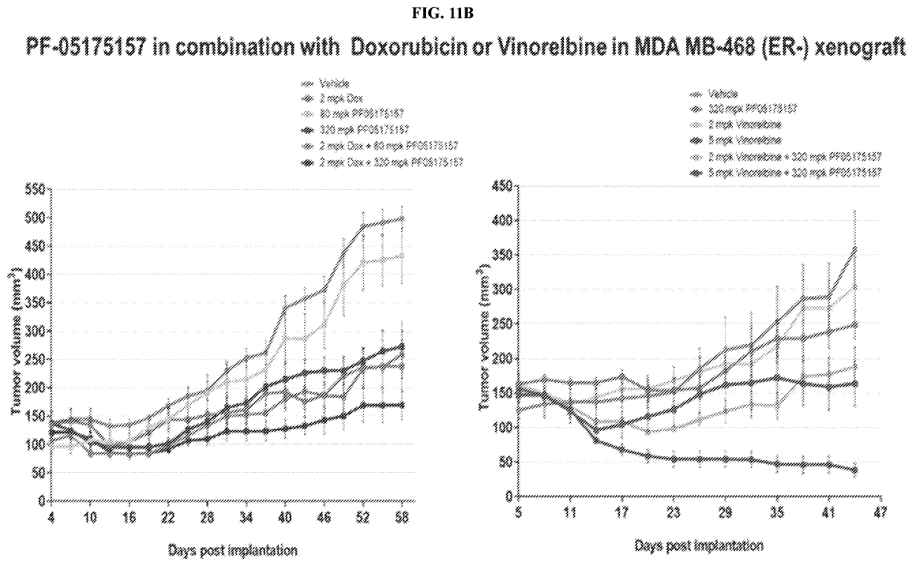

Combinatorial experiments with other agents that suggest additive effects with several different agents in cell line dependent manner (FIGS. 10A-10I). Dose-response curves were generated with administration of PF-05175157 alone and in combination with each of ABT-510, carboplatin. Danshensu, Doxorubicin, Gemcitabine, JZL184, LOXO-101. Metformin, and TVB-2640.

Additionally, the efficacy of PF-05175157 as single agent and in combination with the anti-estrogen fulvestrant and the fatty acid synthase (FASN) inhibitor TBV-3166 in the ER+ human breast cancer cell line, MCF7 and its fulvestrant resistant subclone (MCF7/FR). PF-05175157 demonstrated significant single agent inhibitory effect in both the parental and fulvestrant resistant cells. An additive effect or synergy was not definitively observed when PF-05175157 was combined with fulvestrant or TBV-3166.

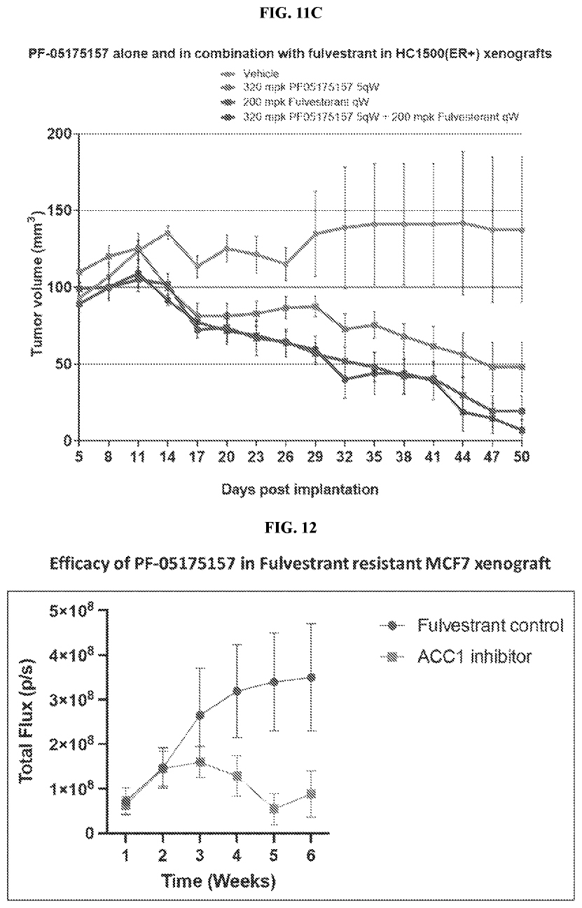

To assess in vivo efficacy of PF-05175157 alone, a human triple negative patient-derived xenograft was treated (20 mg/kg oral gavage twice daily) and tumor growth curves were compared to methylcellulose oral gavage controls. Single agent oral PF-05175157 resulted in 30% growth inhibition (FIG. 11A). To further assess in vivo efficacy of PF-05175157 alone and in combination with doxorubicin or vinorelbine, MDA-MB-468 human triple negative breast cancer cell line xenografts in mice were administered 80 mg/kg and 320 mg/kg PF-05175157 intraperitoneally once daily alone, or with respective chemotherapy and/or chemotherapeutic in cohorts of 6 mice. Intraperitoneal PF-05175157 showed dose dependent single agent growth inhibition and increased the efficacy of low dose doxorubicin and vinorelbine (FIG. 11B).

Subsequently, PF-05175157 was assessed in a human estrogen receptor positive HC 1500 cell line xenograft. Significant single agent growth inhibition (320 mg/kg intraperitoneally daily×5 days followed by 2 days off and repeat every 7 days) was observed in this model (FIG. 11C). A different a human estrogen receptor positive cell line xenograft that comprised fulvestrant resistant MCF7 cells was also used to assess single agent PF05175157 activity. Significant single agent activity was observed (FIG. 12). These results indicate that PF05175157 has a broad antitumor activity in both estrogen receptor positive and negative in vivo breast cancer models. Efficacy does not depend on continued estrogen signaling.

Next, in vitro cytotoxicity assays were performed in 15 breast cancer cell lines and in normal mammary epithelial HMEC cells to test for synergy of PF05157157 with alpelisib, docetaxel, doxorubicin, everolimus, iniparib, neratinib, and TEPP46 (PKM2 and PKLR activator). The effects of these combinations on apoptosis and cell cycle progression was examined. Next, in vivo single agent activity was assessed in a triple negative patient derived (PDX) model (J000102184) in NSG™ mice and in MDAMB468 xenografts implanted into Rag2/IL2RG double knockout mice. RNA sequencing and metabolomic profiling of cells treated with PF05157157 was performed to study metabolic and transcriptomic effects of the drug.

PF05175157 induced time and dose dependent growth inhibition in all but 1 of the 15 cancer cell lines. The estimated EC₅₀ after 72 h exposure ranged from 0.95 to 76 μg/mL in T47D and BT549 cells, respectively (Cmax of 20 μg/mL can be achieved in human serum). There was no significant inhibitory effect on HMEC cells. In cancer cell lines, the percentage of apoptotic cells increased from 4% to 8% in BT474 and from 7.7% to 17.8% in MDMBA468 cells upon treatment with the compound, and there was a trend towards G2/M cell cycle arrest in both cell lines after 72 hours of exposure (10 μg/mL). In drug combination experiments, PF05175157 added to iniparib, or to the PKM2 activator, TEPP46, decreased cell viability compared to single agent therapies in several cell lines. PF05175157 significantly delayed tumor growth compared to vehicle, when administered orally (20 mg/kg gavage BID) in a TNBC PDX model (median tumor volume after 33 days: 334.1 mm³ in PF05175157-treated vs. 490.5 mm³ in methylcellulose-treated mice; p=3.39e-7) and intraperitoneally (20 mg/kg in DMSO) in an MDAMB468 xenograft model (median tumor volume after 40 days: 244.5 mm³ in PF05175157-treated vs. 303.3 mm³ in DMSO-treated mice; p<0.05). Transcriptomic and metabolic profiling of MDAMB468 and BT474 cells treated with 10 μg/ml PF05175157 for 6 and 24 hours revealed activation of immune signaling, epigenetic regulation and DNA damage repair pathways along with down-regulation of a broad range of metabolic pathways.

The small molecule ACC inhibitor, PF05175157, has significant single agent in vitro and in vivo growth inhibitory effect on a range of breast cancer cell lines at concentrations that can be achieved in human serum. It showed synergy with iniparib and another metabolic inhibitor (TEPP46). Targeting de novo fatty acid synthesis by inhibiting ACC is a promising therapeutic strategy.

### Enumerated Embodiments

The following exemplary embodiments are provided, the numbering of which is not to be construed as designating levels of importance:
Embodiment 1 provides a method of treating, preventing, and/or ameliorating cancer in a subject in need thereof,
the method comprising administering to the subject a dose of about 100 to about 600 mg of PF-05175157, or an equimolar amount of a pharmaceutically acceptable salt or solvate thereof.
Embodiment 2 provides the method of Embodiment 1, wherein the cancer is of the breast, bladder, blood, bone, bone marrow, brain, colon, esophagus, gastrointestine, gum, head, kidney, liver, lung, nasopharynx, neck, ovary, prostate, skin, stomach, testis, tongue, pancreas, and/or uterus.
Embodiment 3 provides the method of Embodiment 1 or 2, wherein the cancer is breast cancer.
Embodiment 4 provides the method of Embodiment 3, wherein the breast cancer is selected from the group consisting of HER2-negative metastatic breast cancer, ER-positive/HER2-negative breast cancer, and metastatic triple negative breast cancer (TNBC).
Embodiment 5 provides the method of any one of Embodiments 1-4, wherein the PF-05175157 is administered orally.
Embodiment 6 provides the method of any one of Embodiments 1-5, wherein one of the following applies:
(a) about 200 mg of the PF-05175157 is administered to the subject twice daily; and
(b) about 300 mg of the PF-05175157 is administered to the subject twice daily.
Embodiment 7 provides the method of Embodiment 6, wherein the daily administration is performed for 14 days with a subsequent period of 7 days without daily administration.
Embodiment 8 provides the method of Embodiment 6, the daily administration is performed for 7 days with a subsequent period of 7 days without daily administration.
Embodiment 9 provides the method of any one of Embodiments 1-8, wherein the subject is further administered at least one additional agent.
Embodiment 10 provides the method of Embodiment 9, wherein the at least one additional agent is selected from the group consisting of abemaciclib. ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL184, kadcyla (ado-trastuzumab emtansine), lapatinib, larotrectinib, letrozole, margetuximab, megestrol, methotrexate, metformin. neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).
Embodiment 11 provides the method of Embodiment 9 or 10, wherein the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.
Embodiment 12 provides the method of any one of Embodiments 9-11, wherein the at least one additional agent is elacestrant, optionally wherein the elacestrant is administered orally.
Embodiment 13 provides the method of Embodiment 12, wherein about 400 mg of the elacestrant is administered to the subject daily.
Embodiment 14 provides the method of any one of Embodiments 9-11, wherein the at least one additional agent is sacituzumab govitecan, optionally wherein the sacituzumab govitecan is administered intravenously.
Embodiment 15 provides the method of Embodiment 14, wherein the subject is administered a dose of about 10 mg/kg of the sacituzumab govitecan daily.
Embodiment 16 provides a method of treating, preventing, and/or ameliorating cancer in a subject in need thereof, the method comprising administering to the subject a therapeutically effective amount of an acetyl-CoA carboxylase 1 and/or 2 inhibitor (ACCi) and at least one additional agent.
Embodiment 17 provides the method of Embodiment 16, wherein the ACCi is at least one selected from the group consisting of PF-05175157, firsocostat, TOFA, ND-646, CMS-121, hACC2-IN-1, Acetyl-CoA Carboxvlase-IN-1, CP-640186. MK-4074, olumacostat glasaretil, fluazifop-P-butyl. CP-640186, ACC1/2-IN-1, S-2E, ACC1/2-IN-2, CP-610431, A-908292, haloxyfop, and propaquizafop.
Embodiment 18 provides the method of Embodiment 16 or 17, wherein the ACCi is PF-05175157.
Embodiment 19 provides the method of any one of Embodiments 16-18, wherein the cancer is of the breast, bladder, blood, bone, bone marrow, brain, colon, esophagus. gastrointestine, gum, head, kidney, liver, lung, nasopharynx, neck, ovary, prostate, skin, stomach, testis, tongue, pancreas, and/or uterus.
Embodiment 20 provides the method of any one of Embodiments 16-19, wherein the cancer is breast cancer.
Embodiment 21 provides the method of Embodiment 20, wherein the breast cancer is selected from the group consisting of HER2-negative metastatic breast cancer, ER-positive/HER2-negative breast cancer, and metastatic triple negative breast cancer (TNBC).
Embodiment 22 provides the method of any one of Embodiments 18-21, wherein the PF-05175157 is administered orally.
Embodiment 23 provides the method of any one of Embodiments 18-22, wherein one of the following applies:
(c) about 200 mg of the PF-05175157 is administered to the subject twice daily; and
(d) about 300 mg of the PF-05175157 is administered to the subject twice daily.
Embodiment 24 provides the method of Embodiment 23, wherein the daily administration is performed for 14 days with a subsequent period of 7 days without daily administration.
Embodiment 25 provides the method of Embodiment 23, the daily administration is performed for 7 days with a subsequent period of 7 days without daily administration.
Embodiment 26 provides the method of any one of Embodiments 16-25, wherein the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL184, kadcvla (ado-trastuzumab emtansine), lapatinib, larotrectinib, letrozole, margetuximab, megestrol, methotrexate, metformin. neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin).
Embodiment 27 provides the method of any one of Embodiments 16-26, wherein the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.
Embodiment 28 provides the method of any one of Embodiments 16-27, wherein the at least one additional agent is elacestrant, optionally wherein the elacestrant is administered orally.
Embodiment 29 provides the method of Embodiment 28, wherein about 400 mg of the elacestrant is administered to the subject daily.
Embodiment 30 provides the method of any one of Embodiments 16-27, wherein the at least one additional agent is sacituzumab govitecan, optionally wherein the sacituzumab govitecan is administered intravenously.
Embodiment 31 provides the method of Embodiment 30, wherein the subject is administered a dose of about 10 mg/kg of the sacituzumab govitecan daily.
Embodiment 32 provides the method of any one of Embodiments 1-31, wherein the subject is a mammal.
Embodiment 33 provides the method of Embodiment 32, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of treating, preventing, or ameliorating cancer in a subject in need thereof,
the method comprising administering to the subject a dose of about 100 to about 600 mg of PF-05175157, or an equimolar amount of a pharmaceutically acceptable salt or solvate thereof.

2. The method of claim 1, wherein the cancer is at least one of the breast, bladder, blood, bone, bone marrow, brain, colon, esophagus, gastrointestine, gum, head, kidney, liver, lung, nasopharynx, neck, ovary, prostate, skin, stomach, testis, tongue, pancreas, and uterus.

3. (canceled)

4. The method of claim 2, wherein the breast cancer is selected from the group consisting of HER2-negative metastatic breast cancer, ER-positive/HER2− negative breast cancer, and metastatic triple negative breast cancer (TNBC).

5. The method of claim 1, wherein the PF-05175157 is administered orally.

6. The method of claim 1, wherein one of the following applies:
(a) about 200 mg of the PF-05175157 is administered to the subject twice daily; and
(b) about 300 mg of the PF-05175157 is administered to the subject twice daily.

7. The method of claim 6, wherein the daily administration is performed for 7 or 14 days, either of which with a subsequent period of 7 days without daily administration.

8. (canceled)

9. The method of claim 1, wherein the subject is further administered at least one additional agent.

10. The method of claim 9, wherein the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL184, kadcyla (ado-trastuzumab emtansine), lapatinib, larotrectinib, letrozole, margetuximab, megestrol, methotrexate, metformin, neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin),
optionally wherein the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.

11. (canceled)

12. The method of claim 9, wherein the at least one additional agent is elacestrant, optionally wherein the elacestrant is administered orally, and optionally wherein about 400 mg of the elacestrant is administered to the subject daily.

13. (canceled)

14. The method of claim 9, wherein the at least one additional agent is sacituzumab govitecan, optionally wherein the sacituzumab govitecan is administered intravenously, and optionally wherein the subject is administered a dose of about 10 mg/kg of the sacituzumab govitecan daily.

15. (canceled)

16. A method of treating, preventing, or ameliorating cancer in a subject in need thereof, the method comprising administering to the subject a therapeutically effective amount of an acetyl-CoA carboxylase 1 or 2 inhibitor (ACCi) and at least one additional agent.

17. The method of claim 16, wherein the ACCi is at least one selected from the group consisting of PF-05175157, firsocostat, TOFA, ND-646, CMS-121, hACC2-IN-1, Acetyl-CoA Carboxylase-IN-1, CP-640186, MK-4074, olumacostat glasaretil, fluazifop-P-butyl, CP-640186, ACC1/2-IN-1, S-2E, ACC1/2-IN-2, CP-610431, A-908292, haloxyfop, and propaquizafop.

18. (canceled)

19. The method of claim 16, wherein the cancer is at least one of the breast, bladder, blood, bone, bone marrow, brain, colon, esophagus, gastrointestine, gum, head, kidney, liver, lung, nasopharynx, neck, ovary, prostate, skin, stomach, testis, tongue, pancreas, and uterus.

20. (canceled)

21. The method of claim 19, wherein the breast cancer is selected from the group consisting of HER2-negative metastatic breast cancer, ER-positive/HER2-negative breast cancer, and metastatic triple negative breast cancer (TNBC).

22. The method of any one of claim 17, wherein the PF-05175157 is administered orally.

23. The method of any one of claim 17, wherein one of the following applies:
(c) about 200 mg of the PF-05175157 is administered to the subject twice daily; and
(d) about 300 mg of the PF-05175157 is administered to the subject twice daily.

24. The method of claim 23, wherein the daily administration is performed for 7 or 14 days either of which with a subsequent period of 7 days without daily administration.

25. (canceled)

26. The method of claim 16, wherein the at least one additional agent is selected from the group consisting of abemaciclib, ABT-510, abraxane, ado-trastuzumab emtansine, trastuzumab-deruxtecan, alpelisib, anastrozole, aredia (pamidronate disodium), aromasin (exemestane), atezolizumab, capecitabine, carboplatin, cyclophosphamide, danshensu, denifanstat (TVB-2640), docetaxel, doxorubicin, elacestrant, enhertu, epirubicin, eribulin, everolimus, exemestane, 5-FU (fluorouracil injection), fam-trastuzumab deruxtecan, fareston (toremifene), femara (letrozole), fulvestrant, gemcitabine, goserelin, herceptin hylecta (trastuzumab and hyaluronidase), ixabepilone, iniparib, JZL184, kadcyla (ado-trastuzumab emtansine), lapatinib, larotrectinib, letrozole, margetuximab, megestrol, methotrexate, metformin, neratinib, olaparib, paclitaxel, palbociclib, pembrolizumab, pertuzumab, hyaluronidase, phesgo, ribociclib, sacituzumab govitecan (Trodelyy), talazoparib, tamoxifen, TEPP-46, tepadina (thiotepa), thiotepa, toremifen, trastuzumab, trexall (methotrexate sodium), tucatinib, verzenio (abemaciclib), vinblastine, xeloda (capecitabine), and zoladex (goserelin),
optionally wherein the at least one additional agent is at least one selected from the group consisting of ABT-510, alpelisib, carboplatin, danshensu, denifanstat (TVB-2640), docetaxel, doxorubicin, elacestrant, everolimus, gemcitabine, iniparib, JZL184, metformin, neratinib, sacituzumab govitecan, and TEPP-46.

27. (canceled)

28. The method of claim 16, wherein the at least one additional agent is elacestrant, optionally wherein the elacestrant is administered orally, and optionally wherein about 400 mg of the elacestrant is administered to the subject daily.

29. (canceled)

30. The method of claim 16, wherein the at least one additional agent is sacituzumab govitecan, optionally wherein the sacituzumab govitecan is administered intravenously, and optionally wherein the subject is administered a dose of about 10 mg/kg of the sacituzumab govitecan daily.

31-33. (canceled)
